(19) United States
(12) Patent Application Publication
(10) Pub. No.: US 2023/0133958 A1
(43) Pub. Date: May 4, 2023

(54) COMPOUNDS USEFUL IN MODULATING EGFR AND PI3K

(71) Applicant: Mekanistic Therapeutics LLC, Ann Arbor, MI (US)

(72) Inventors: Christopher E. Whitehead, Ypsilanti, MI (US); Judith S. Leopold, Ann Arbor, MI (US)

(21) Appl. No.: 17/970,443

(22) Filed: Oct. 20, 2022

Related U.S. Application Data
(60) Provisional application No. 63/257,756, filed on Oct. 20, 2021.

(51) Int. Cl. C07D 401/04

(52) U.S. Cl. CPC C07D 401/04

(57) ABSTRACT

The present invention relates to the field of medicinal chemistry. In particular, the invention relates to a new class of small-molecules having a quinazoline structure or a quinoline structure according to Formula I, wherein L, R¹, R², X¹, X², and X³ are described herein,

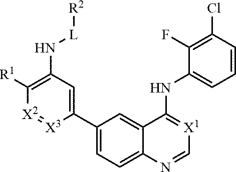

which function as dual inhibitors of EGFR proteins and PI3K proteins. The invention further relates to their use as therapeutics for the treatment of EGFR and/or PI3K mediated diseases or conditions.

### CROSS REFERENCE TO RELATED APPLICATION

This application claims the benefit of U.S. Provisional Application No. 63/257,756, filed on Oct. 20, 2021. The entire contents of the aforementioned application is hereby incorporated by reference in its entirety.

### STATEMENT REGARDING FEDERALLY SPONSORED RESEARCH OR DEVELOPMENT

This invention was made with government support under Grant No. R44CA213715 awarded by the National Institutes of Health. The government has certain rights in the invention.

### TECHNICAL FIELD OF THE INVENTION

The present invention relates to the field of medicinal chemistry. In particular, the invention relates to a new class of small-molecules having a quinazoline structure or a quinoline structure which function as dual inhibitors of EGFR proteins and PI3K proteins. The invention further relates to their use as therapeutics for the treatment of EGFR and/or PI3K mediated diseases or conditions.

### BACKGROUND

The Epidermal Growth Factor Receptor (EGFR) is a member of the ErbB family of receptors, a subfamily of four closely related receptor tyrosine kinases: EGFR (ErbB-1), HER2/neu (ErbB-2), Her 3 (ErbB-3) and Her 4 (ErbB-4). Mutations affecting EGFR expression or activity could result in cancer.

Mutations that lead to EGFR overexpression have been associated with a number of cancers, including adenocarcinoma of the lung (40% of cases), anal cancers, glioblastoma (50%) and epithelian tumors of the head and neck (80-100%). These somatic mutations involving EGFR lead to its constant activation, which produces uncontrolled cell division. In glioblastoma a specific mutation of EGFR, called EGFRvIII, is often observed. Mutations, amplifications or misregulations of EGFR or family members are implicated in about 30% of all epithelial cancers.

Phosphoinositide 3-kinase (PI3K) proteins are a family of related intracellular signal transducer enzymes capable of phosphorylating the 3 position hydroxyl group of the inositol ring of phosphatidylinositol (PtdIns). The pathway, with oncogene PIK3CA and tumor suppressor gene PTEN, is implicated in the sensitivity of cancer tumors to insulin and IGF1, and in calorie restriction.

The class IA PI3K p110α is mutated in many cancers. Many of these mutations cause the kinase to be more active. It is the single most mutated kinase in glioblastoma, the most malignant primary brain tumor. The PtdIns(3,4,5)P₃ phosphatase PTEN that antagonises PI3K signaling is absent from many tumors. In addition, the epidermal growth factor receptor EGFR that functions upstream of PI3K is mutationally activated or overexpressed in cancer. Hence, PI3K activity contributes significantly to cellular transformation and the development of cancer.

Colorectal cancer is the third most prevalent malignancy in the United States with approximately 145,000 new diagnoses and 56,000 deaths estimated for 2005 (see, e.g., Cancer Facts and Figures 2005, Surveillance Research (Washington, D.C.: American Cancer Society, Inc.), 2005). Surgery is the mainstay of treatment for colorectal cancer but recurrence is frequent. Colorectal cancer has proven resistant to chemotherapy, although limited success has been achieved using a combination of 5-fluorouracil and levamisole. Surgery has had the largest impact on survival and, in some patients with limited disease, achieves a cure. However, surgery removes bulk tumor, leaving behind microscopic residual disease which ultimately results in recurrence.

### SUMMARY OF THE INVENTION

In one aspect, the invention includes a compound of Formula I:

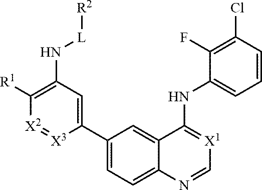

or a pharmaceutically acceptable salt thereof, wherein
X¹ is selected from N or CR³, wherein R³ can be hydrogen, CN, NO₂, NH₂, OH, COOH, halo, C₁₋₆ alkyl, O—C₁₋₆ alkyl, C(O)C₁₋₆ alkyl, and C(O)OC₁₋₆ alkyl, wherein each C₁₋₆ alkyl is optionally substituted;
X² and X³ are each independently selected from N and CH;
L is a bond or a bivalent linking moiety selected from C₁₋₆ alkylene, —C(O)—, —C(O)O—, —S—, —O—, —NR⁴—, —S(O)₂—, —NHS(O)₂—, and —C(O)NH—;
R¹ is selected from hydrogen, C₁₋₆ alkyl, C₃₋₆ cycloalkyl, a 3-7 membered heterocycloalkyl, phenyl, a 5-6 membered heteroaryl, CN, NO₂, NH₂, OH, COOH, halo, O-C₁₋₆ alkyl, C(O)C₁₋₆ alkyl, and C(O)OC₁₋₆ alkyl, wherein each C₁₋₆ alkyl, C₃₋₆ cycloalkyl, 3-7 membered heterocycloalkyl, phenyl, and 5-6 membered heteroaryl is optionally and independently substituted; and
R² is selected from hydrogen, a C₁₋₆ alkyl, a C₃₋₆ cycloalkyl, a 3-7 membered heterocycloalkyl, phenyl, and a 5-6 membered heteroaryl, each of which is optionally and independently substituted.

In another aspect, the invention includes a pharmaceutical composition comprising a compound, or salt thereof, as described herein, and a pharmaceutically acceptable excipient.

In still another aspect, the invention includes a method of treating, ameliorating, or preventing a EGFR and/or PI3K mediated disease or condition in a patient, comprising administering to said patient a therapeutically effective amount of a compound, or salt thereof, as described herein, or a pharmaceutical composition as described herein.

In still another aspect, the invention includes a kit comprising a compound, or salt thereof, as described herein, and instructions for administering said compound to a patient having a EGFR and/or PI3K mediated disease or condition.

### DETAILED DESCRIPTION OF THE INVENTION

### Definitions

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry,” Thomas Sorrell, University Science Books, Sausalito: 1999, and “March’s Advanced Organic Chemistry,” 5th Ed., Ed.: Smith, M.B. and March, J., John Wiley & Sons, New York: 2001, the entire contents of which are hereby incorporated by reference.

As used herein, an “alkyl” group refers to a saturated aliphatic hydrocarbon group containing 1-12 (e.g., 1-8, 1-6, or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, or heterocycloaliphaticamino], sulfonyl [e.g., aliphatic—SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “aryl” group used alone or as part of a larger moiety as in “aralkyl,” “aralkoxy,” or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl [e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphatic—SO₂— or amino—SO₂—]; sulfinyl [e.g., aliphatic—S(O)— or cycloaliphatic—S(O)—]; sulfanyl [e.g., aliphatic—S—]; cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl [e.g., mono-, di (such as p,m—dihaloaryl), and (trihalo)aryl]; (carboxy)aryl [e.g., (alkoxycarbonyl)aryl, ((aralkyl)carbonyloxy)aryl, and (alkoxycarbonyl)aryl]; (amido)aryl [e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl]; aminoaryl [e.g., ((alkylsulfonyl)amino)aryl or ((dialkyl)amino)aryl]; (cyanoalkyl)aryl; (alkoxy)aryl; (sulfamoyl)aryl [e.g., (aminosulfonyl)aryl]; (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, or ((aminocarbonyl)cycloalkyl)cycloalkyl.

A cycloalkyl group can be optionally substituted with one or more substituents such as phospho, aliphatic [e.g., alkyl, alkenyl, or alkynyl], cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino], nitro, carboxy [e.g., HOOC-, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], cyano, halo, hydroxy, mercapto, sulfonyl [e.g., alkyl—SO₂— and aryl—SO₂—], sulfinyl [e.g., alkyl—S(O)—], sulfanyl [e.g., alkyl—S—], sulfoxy, urea, thiourea, sulfamoyl, sulfamide, oxo, or carbamoyl.

As used herein, a “heterocycloalkyl” group refers to a 3-10 membered mono- or bicylic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4—dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety to form structures, such as tetrahydroisoquinoline, that would be categorized as heteroaryls.

A heterocycloalkyl group can be optionally substituted with one or more substituents such as phospho, aliphatic [e.g., alkyl, alkenyl, or alkynyl], cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, or (heteroaraliphatic)carbonylamino], nitro, carboxy [e.g., HOOC-, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], nitro, cyano, halo, hydroxy, mercapto, sulfonyl [e.g., alkylsulfonyl or arylsulfonyl], sulfinyl [e.g., alkylsulfinyl], sulfanyl [e.g., alkylsulfanyl], sulfoxy, urea, thiourea, sulfamoyl, sulfamide, oxo, or carbamoyl.

A “heteroaryl” group, as used herein, refers to a monocyclic, bicyclic, or tricyclic ring system having 4 to 15 ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophene-yl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl,cinnolyl, phthalazyl, quinazolyl, quinoxalyl, isoquinolyl, 4H-quinolizyl, benzo-1,2,5-thiadiazolyl, or 1,8-naphthyridyl.

Without limitation, monocyclic heteroaryls include furyl, thiophene-yl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl [ e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphaticsulfonyl or aminosulfonyl]; sulfinyl [e.g., aliphaticsulfinyl]; sulfanyl [e.g., aliphaticsulfanyl]; nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl [e.g., mono-and di-(halo)heteroaryl]; (carboxy)heteroaryl [e.g., (alkoxycarbonyl)heteroaryl]; cyanoheteroaryl; aminoheteroaryl [e.g., ((alkylsulfonyl)amino)heteroaryl and ((dialkyl)amino)heteroaryl]; (amido)heteroaryl [e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl]; (cyanoalkyl)heteroaryl; (alkoxy)heteroaryl; (sulfamoyl)heteroaryl [e.g., (aminosulfonyl)heteroaryl]; (sulfonyl)heteroaryl [e.g., (alkylsulfonyl)heteroaryl]; (hydroxyalkyl)heteroaryl; (alkoxyalkyl)heteroaryl; (hydroxy)heteroaryl; ((carboxy)alkyl)heteroaryl; (((dialkyl)amino)alkyl]heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl [e.g., (alkylcarbonyl)heteroaryl]; (alkyl)heteroaryl; or (haloalkyl)heteroaryl [e.g., trihaloalkylheteroaryl].

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

As used herein, an “alkoxy” group refers to an alkyl—O— group where “alkyl” has been defined previously.

As used herein, a “haloalkyl” group refers to an alkyl group substituted with 1-3 halogen. For instance, the term haloalkyl includes the group —CF₃.

As used herein, a “carbonyl” refers to —C(O)—.

As used herein, an “oxo” refers to ═O.

As used herein, the term “vicinal” generally refers to the placement of substituents on a group that includes two or more carbon atoms, wherein the substituents are attached to adjacent carbon atoms.

As used herein, the term “geminal” generally refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{x}O(O)C-alkyl, is an example of a carboxy group used terminally. A group is internal when the group is present in the middle of a substituent of the chemical structure. Alkylcarboxy (e.g., alkyl—C(O)O— or alkyl—OC(O)—) and alkylcarboxyaryl (e.g., alkyl-C(O)O-aryl- or alkyl-O(CO)-aryl-) are examples of carboxy groups used internally.

The phrase “optionally substituted” is used herein interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R¹, X, L, X¹, X², X³, X⁴, X⁵, X⁶ and other variables contained in Formula (I), (II), and (II-A) described herein encompass specific groups, such as alkyl and aryl. Unless otherwise noted, each of the specific groups for the variables R¹, X, L, X¹, X², X³, X⁴, X⁵, X⁶ and other variables contained therein can be optionally substituted with one or more substituents described herein. Each substituent of a specific group is further optionally substituted with one to three of halo, cyano, oxo, alkoxy, hydroxy, amino, nitro, aryl, cycloaliphatic, heterocycloaliphatic, heteroaryl, haloalkyl, and alkyl. For instance, an alkyl group can be substituted with alkylsulfanyl and the alkylsulfanyl can be optionally substituted with one to three of halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkxoy groups can form a ring together with the atom(s) to which they are bound.

As used herein, the term “substituted,” whether preceded by the term “optionally” or not, refers generally to the replacement of hydrogen atoms in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

As used herein, the phrase “stable or chemically feasible” refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

Unless otherwise stated, structures depicted herein also are meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein also are meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays, or as therapeutic agents.

It is noted that the use of the descriptors “first,” “second,” “third,” or the like is used to differentiate separate elements (e.g., solvents, reaction steps, processes, reagents, or the like) and may or may not refer to the relative order or relative chronology of the elements described.

The term “anticancer agent” as used herein, refer to any therapeutic agents (e.g., chemotherapeutic compounds and/or molecular therapeutic compounds), antisense therapies, radiation therapies, or surgical interventions, used in the treatment of hyperproliferative diseases such as cancer (e.g., in mammals, e.g.., in humans).

The term “prodrug” as used herein, refers to a pharmacologically inactive derivative of a parent “drug” molecule that requires biotransformation (e.g., either spontaneous or enzymatic) within the target physiological system to release, or to convert (e.g., enzymatically, physiologically, mechanically, electromagnetically) the prodrug into the active drug. Prodrugs are designed to overcome problems associated with stability, water solubility, toxicity, lack of specificity, or limited bioavailability. Exemplary prodrugs comprise an active drug molecule itself and a chemical masking group (e.g., a group that reversibly suppresses the activity of the drug). Some prodrugs are variations or derivatives of compounds that have groups cleavable under metabolic conditions. Prodrugs can be readily prepared from the parent compounds using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309-396; Burger’s Medicinal Chemistry and Drug Discovery, 5th Ed., M. Wolff (ed.), John Wiley & Sons, 1995, particularly Vol. 1 and pp. 172-178 and pp. 949-982; Pro-Drugs as Novel Delivery Systems, T. Higuchi and V. Stella (eds.), Am. Chem. Soc., 1975; and Bioreversible Carriers in Drug Design, E. B. Roche (ed.), Elsevier, 1987.

Exemplary prodrugs become pharmaceutically active in vivo or in vitro when they undergo solvolysis under physiological conditions or undergo enzymatic degradation or other biochemical transformation (e.g., phosphorylation, hydrogenation, dehydrogenation, glycosylation). Prodrugs often offer advantages of water solubility, tissue compatibility, or delayed release in the mammalian organism. (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, CA (1992)). Common prodrugs include acid derivatives such as esters prepared by reaction of parent acids with a suitable alcohol (e.g., a lower alkanol) or esters prepared by reaction of parent alcohol with a suitable carboxylic acid, (e.g., an amino acid), amides prepared by reaction of the parent acid compound with an amine, basic groups reacted to form an acylated base derivative (e.g., a lower alkylamide), or phosphorus-containing derivatives, e.g., phosphate, phosphonate, and phosphoramidate esters, including cyclic phosphate, phosphonate, and phosphoramidate (see, e.g., U.S. Pat. Application Publication No. US 2007/0249564 A1; herein incorporated by reference in its entirety).

The term “pharmaceutically acceptable salt” as used herein, refers to any salt (e.g., obtained by reaction with an acid or a base) of a compound of the present invention that is physiologically tolerated in the target patient (e.g., a mammal). Salts of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, mesylate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “solvate” as used herein, refers to the physical association of a compound of the invention with one or more solvent molecules, whether organic or inorganic. This physical association often includes hydrogen bonding. In certain instances, the solvate is capable of isolation, for example, when one or more solvate molecules are incorporated in the crystal lattice of the crystalline solid. “Solvate” encompasses both solution-phase and isolable solvates. Exemplary solvates include hydrates, ethanolates, and methanolates.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, in one embodiment, a therapeutically effective amount will refer to the amount of a therapeutic agent that decreases the rate of tumor growth, decreases tumor mass, decreases the number of metastases, increases time to tumor progression, or increases survival time by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The terms “sensitize” and “sensitizing,” as used herein, refer to making, through the administration of a first agent (e.g., a quinazoline compound of the invention), an animal or a cell within an animal more susceptible, or more responsive, to the biological effects (e.g., promotion or retardation of an aspect of cellular function including, but not limited to, cell division, cell growth, proliferation, invasion, angiogenesis, necrosis, or apoptosis) of a second agent. The sensitizing effect of a first agent on a target cell can be measured as the difference in the intended biological effect (e.g., promotion or retardation of an aspect of cellular function including, but not limited to, cell growth, proliferation, invasion, angiogenesis, or apoptosis) observed upon the administration of a second agent with and without administration of the first agent. The response of the sensitized cell can be increased by at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least 300%, at least about 350%, at least about 400%, at least about 450%, or at least about 500% over the response in the absence of the first agent.

The term “dysregulation of apoptosis,” as used herein, refers to any aberration in the ability of (e.g., predisposition) a cell to undergo cell death via apoptosis. Dysregulation of apoptosis is associated with or induced by a variety of conditions, non-limiting examples of which include, autoimmune disorders (e.g., systemic lupus erythematosus, rheumatoid arthritis, graft-versus-host disease, myasthenia gravis, or Sjogren’s syndrome), chronic inflammatory conditions (e.g., psoriasis, asthma or Crohn’s disease), hyperproliferative disorders (e.g., tumors, B cell lymphomas, or T cell lymphomas), viral infections (e.g., herpes, papilloma, or HIV), and other conditions such as osteoarthritis and atherosclerosis.

The term “hyperproliferative disease,” as used herein, refers to any condition in which a localized population of proliferating cells in a patient is not governed by the usual limitations of normal growth. Examples of hyperproliferative disorders include tumors, neoplasms, lymphomas and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The pathological growth of activated lymphoid cells often results in an autoimmune disorder or a chronic inflammatory condition. As used herein, the term “autoimmune disorder” refers to any condition in which an organism produces antibodies or immune cells which recognize the organism’s own molecules, cells or tissues. Non-limiting examples of autoimmune disorders include autoimmune hemolytic anemia, autoimmune hepatitis, Berger’s disease or IgA nephropathy, celiac sprue, chronic fatigue syndrome, Crohn’s disease, dermatomyositis, fibromyalgia, graft versus host disease, Grave’s disease, Hashimoto’s thyroiditis, idiopathic thrombocytopenia purpura, lichen planus, multiple sclerosis, myasthenia gravis, psoriasis, rheumatic fever, rheumatic arthritis, scleroderma, Sjogren’s syndrome, systemic lupus erythematosus, type 1 diabetes, ulcerative colitis, vitiligo, and the like.

The term “neoplastic disease,” as used herein, refers to any abnormal growth of cells being either benign (non-cancerous) or malignant (cancerous).

The term “normal cell,” as used herein, refers to a cell that is not undergoing abnormal growth or division. Normal cells are non-cancerous and are not part of any hyperproliferative disease or disorder.

The term “anti-neoplastic agent,” as used herein, refers to any compound that retards the proliferation, growth, or spread of a targeted (e.g., malignant) neoplasm.

The terms “prevent,” “preventing,” and “prevention,” as used herein, refer to a decrease in the occurrence of pathological cells (e.g., hyperproliferative or neoplastic cells) in a patient. The prevention may be complete, e.g., the total absence of pathological cells in a subject. The prevention may also be partial, such that the occurrence of pathological cells in a subject is less than that which would have occurred without the present invention.

The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington’s Pharmaceutical Sciences, Mack Publishing Co., Easton, PA, 19th ed. 1995.

### Embodiments

In one aspect, the invention includes a compound of Formula I:

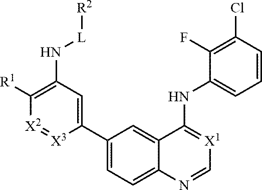

or a pharmaceutically acceptable salt thereof, wherein
X¹ is selected from N or CR³, wherein R³ can be hydrogen, CN, NO₂, NH₂, OH, COOH, halo, C₁₋₆ alkyl, O—C₁₋₆ alkyl, C(O)C₁₋₆ alkyl, and C(O)OC₁₋₆ alkyl, wherein each C₁₋₆ alkyl is optionally substituted;
X² and X³ are each independently selected from N and CH;
L is a bond or a bivalent linking moiety selected from C₁₋₆ alkylene, —C(O)—, —C(O)O—, —S—, —O—, —NR⁴—, —S(O)₂—, —NHS(O)₂—, and —C(O)NH—;
R¹ is selected from hydrogen, C₁₋₆ alkyl, C₃₋₆ cycloalkyl, a 3-7 membered heterocycloalkyl, phenyl, a 5-6 membered heteroaryl, CN, NO₂, NH₂, OH, COOH, halo, O-C₁₋₆ alkyl, C(O)C₁₋₆ alkyl, and C(O)OC₁₋₆ alkyl, wherein each C₁₋₆ alkyl, C₃₋₆ cycloalkyl, 3-7 membered heterocycloalkyl, phenyl, and 5-6 membered heteroaryl is optionally and independently substituted; and
R² is selected from hydrogen, a C₁₋₆ alkyl, a C₃₋₆ cycloalkyl, a 3-7 membered heterocycloalkyl, phenyl, and a 5-6 membered heteroaryl, each of which is optionally and independently substituted.

In another aspect, the invention includes a compound of Formula II:

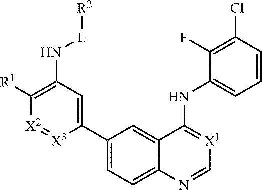

or a pharmaceutically acceptable salt thereof, wherein
X¹ is selected from N or CR³, wherein R³ can be hydrogen, CN, NO₂, NH₂, OH, COOH, halo, C₁₋₆ alkyl, O—C₁₋₆ alkyl, C(O)C₁₋₆ alkyl, and C(O)OC₁₋₆ alkyl, wherein each C₁₋₆ alkyl is optionally substituted;
X² and X³ are each independently selected from N and CH;
L is a bivalent linking moiety selected from C₁₋₆ alkylene, —C(O)—, —C(O)O—, —S—, —O—, —NR⁴—, —S(O)₂—, —NHS(O)₂—, and —C(O)NH—;
R¹ is selected from hydrogen, C₁₋₆ alkyl, C₃₋₆ cycloalkyl, a 3-7 membered heterocycloalkyl, phenyl, a 5-6 membered heteroaryl, CN, NO₂, NH₂, OH, COOH, halo, O-C₁₋₆ alkyl, C(O)C₁₋₆ alkyl, and C(O)OC₁₋₆ alkyl, wherein each C₁₋₆ alkyl, C₃₋₆ cycloalkyl, 3-7 membered heterocycloalkyl, phenyl, and 5-6 membered heteroaryl is optionally and independently substituted;
R² is selected from a C₁₋₆ alkyl, a C₃₋₆ cycloalkyl, a 3-7 membered heterocycloalkyl, phenyl, and a 5-6 membered heteroaryl, each of which is optionally and independently substituted.

In one embodiment, R³ can be hydrogen, NO₂, NH₂, OH, COOH, halo, C₁₋₆ alkyl, O-C₁₋₆ alkyl, C(O)C₁₋₆ alkyl, and C(O)OC₁₋₆ alkyl, wherein each C₁₋₆ alkyl is optionally substituted.

In another embodiment, R³ can be hydrogen, halo, or C₁₋₆ alkyl, wherein each C₁₋₆ alkyl is optionally substituted.

In a further embodiment, R³ can be hydrogen, halo, methyl, ethyl, propyl, isopropyl, butyl, or tert-butyl.

In one embodiment, R³ can be hydrogen, CN, NH₂, OH, COOH, halo, C₁₋₆ alkyl, O-C₁₋₆ alkyl, C(O)C₁₋₆ alkyl, and C(O)OC₁₋₆ alkyl, wherein each C₁₋₆ alkyl is optionally substituted.

In another embodiment, R³ can be CN, hydrogen, halo, or C₁₋₆ alkyl, wherein each C₁₋₆ alkyl is optionally substituted.

In a further embodiment, R³ can be hydrogen, CN, halo, methyl, ethyl, propyl, isopropyl, butyl, or tert-butyl.

In another further embodiment, R³ can be CN.

In some embodiments, X¹ is CR³. In other embodiments, X¹ is N.

In one embodiment, at least one of X² and X³ is N.

In a further embodiment, one of X² and X³ is N, and the other of X² and X³ is CH.

In still a further embodiment, X² is N, and X³ is CH.

In another embodiment, both of X² and X³ are CH.

In one embodiment, R¹ is selected from hydrogen, C₁₋₆ alkyl, C₃₋₆ cycloalkyl, a 3-7 membered heterocycloalkyl, phenyl, a 5-6 membered heteroaryl, CN, NO₂, NH₂, OH, COOH, halo, O-C₁₋₆ alkyl, C(O)C₁₋₆ alkyl, and C(O)OC₁₋₆ alkyl, wherein each C₁₋₆ alkyl, C₃₋₆ cycloalkyl, 3-7 membered heterocycloalkyl, phenyl, and 5-6 membered heteroaryl is optionally and independently substituted.

In another embodiment, R¹ is selected from hydrogen, C₁₋₆ alkyl, CN, NO₂, NH₂, OH, COOH, halo, O-C₁₋₆ alkyl, C(O)C₁₋₆ alkyl, and C(O)OC₁₋₆ alkyl, wherein each C₁₋₆ alkyl, is optionally and independently substituted with one or more of oxo, halo, OH, COOH, CONH₂, or CN.

In one embodiment, R¹ is selected from hydrogen, C₁₋₆ alkyl, halo, and O-C₁₋₆ alkyl, wherein each C₁₋₆ alkyl, is optionally and independently substituted with one or more of oxo, halo, OH, or CN.

In another embodiment, R¹ is selected from hydrogen, C₁₋₆ alkyl, C₁₋₆ haloalkyl, halo, and O-C₁₋₆ alkyl.

In a further embodiment, R¹ is selected from hydrogen, methyl, ethyl, propyl, methoxy, trifluoromethyl, and halo.

In still a further embodiment, R¹ is selected from hydrogen, methyl, methoxy, trifluoromethyl, and chloro.

In one embodiment, L is a bivalent linking moiety selected from C₁₋₆ alkylene, —C(O)—, —C(O)O—, —S—, —O—, —NR⁴—, —S(O)₂—, —NHS(O)₂—, and —C(O)NH—.

In another embodiment, L is a bond or a bivalent linking moiety selected from —C(O)—, —C(O)O—, —S(O)₂—, —NHS(O)₂—, and —C(O)NH—.

In one embodiment, L is a bivalent linking moiety selected from C₁₋₆ alkylene, —C(O)—, —C(O)O—, —S—, —O—, —NR⁴—, —S(O)₂—, —NHS(O)₂—, and —C(O)NH—.

In another embodiment, L is a bond or a bivalent linking moiety selected from —C(O)—, —C(O)O—, —S(O)₂—, —NHS(O)₂—, and —C(O)NH—.

In a further embodiment, L is a bivalent linking moiety selected from —C(O)—, —C(O)O—, —S(O)₂—, —NHS(O)₂—, and —C(O)NH—.

In still a further embodiment, L is a bivalent linking moiety selected from —C(O)—, and —S(O)₂—.

In yet a further embodiment, L is —S(O)_{2—}.

In one embodiment, R² is selected from a C₁₋₆ alkyl, a C₃₋₆ cycloalkyl, and a 3-7 membered heterocycloalkyl, each of which is optionally and independently substituted with one or more of halo, CN, OH, COOH, —N(R^{a})₂, wherein each R^{a} is H or C₁₋₆ alkyl, a C₁₋₆ alkyl, a C₃₋₆ cycloalkyl, a 3-7 membered heterocycloalkyl, phenyl, or a 5-6 membered heteroaryl, each of which is optionally and independently substituted with C₁₋₆ alkyl, halo or OH.

In another embodiment, R² is selected from a C₁₋₆ alkyl, optionally and independently substituted with one or more of halo, OH, —NH₂, —N(H)CH₃, —N(CH₃)₂, a C₁₋₆ alkyl, a C₃₋₆ cycloalkyl, a 3-7 membered heterocycloalkyl, phenyl, or a 5-6 membered heteroaryl, each of which is optionally and independently substituted with C₁₋₆ alkyl, halo or OH.

In one embodiment, R² is selected from hydrogen, a C₁₋₆ alkyl, a C₃₋₆ cycloalkyl, and a 3-7 membered heterocycloalkyl, each of which is optionally and independently substituted with one or more of halo, CN, OH, COOH, —N(R^{a})₂, wherein each R^{a} is H or C₁₋₆ alkyl, a C₁₋₆ alkyl, a C₃₋₆ cycloalkyl, a 3-7 membered heterocycloalkyl, phenyl, or a 5-6 membered heteroaryl, each of which is optionally and independently substituted with C₁₋₆ alkyl, halo or OH.

In another embodiment, R² is selected from hydrogen, a C₁₋₆ alkyl, optionally and independently substituted with one or more of halo, OH, —NH₂, —N(H)CH₃, —N(CH₃)₂, a C₁₋₆ alkyl, a C₃₋₆ cycloalkyl, a 3-7 membered heterocycloalkyl, phenyl, or a 5-6 membered heteroaryl, each of which is optionally and independently substituted with C₁₋₆ alkyl, halo or OH.

In another embodiment, R² is a C₁₋₆ alkyl, optionally and independently substituted with one or more of —NH₂, —N(H)CH₃, —N(CH₃)₂, or a 3-7 membered heterocycloalkyl, each of which is optionally and independently substituted with C₁₋₆ alkyl.

In a further embodiment, R² is a methyl, ethyl, or propyl.

In still a further embodiment, R² is a methyl.

In another embodiment, R² is a C₁₋₆ alkyl, substituted with —NH₂, —N(H)CH₃, —N(CH₃)₂, or a 3-7 membered heterocycloalkyl, which is optionally substituted with C₁₋₆ alkyl.

In a further embodiment, R² is a C₁₋₆ alkyl, substituted with —N(CH₃)₂.

In another embodiment, R² is a C₁₋₆ alkyl, substituted with morpholine or N-methylpiperizine.

In a further embodiment, R² is selected from

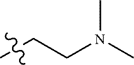

,

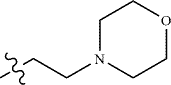

, and

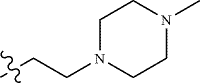

In one embodiment, the compound of Formula I is a compound of Formula III:

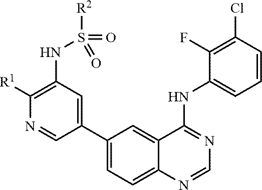

or a pharmaceutically acceptable salt thereof, wherein R¹ and R² are defined herein.

In one embodiment, the compound of Formula I is selected from the compounds listed in Table 1.

TABLE 1

Compounds of Formula I

| | |
|---|---|
| 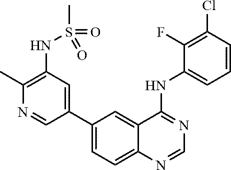 [SMILES: Cc1ncc(-c2ccc3ncnc(Nc4cccc(Cl)c4F)c3c2)cc1NS(C)(=O)=O] | 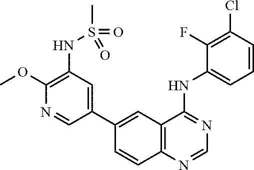 [SMILES: COc1ncc(-c2ccc3ncnc(Nc4cccc(Cl)c4F)c3c2)cc1NS(C)(=O)=O] |
| Compound 1 | Compound 2 |
| 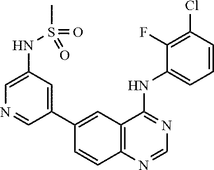 [SMILES: CS(=O)(=O)Nc1cncc(-c2ccc3ncnc(Nc4cccc(Cl)c4F)c3c2)c1] | 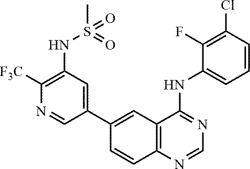 [SMILES: CS(=O)(=O)Nc1cc(-c2ccc3ncnc(Nc4cccc(Cl)c4F)c3c2)cnc1C(F)(F)F] |
| Compound 3 | Compound 4 |
| 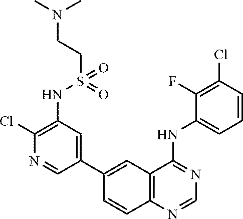 [SMILES: CN(C)CCS(=O)(=O)Nc1cc(-c2ccc3ncnc(Nc4cccc(Cl)c4F)c3c2)cnc1Cl] | 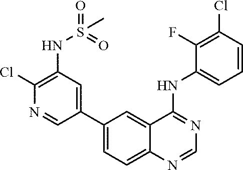 [SMILES: CS(=O)(=O)Nc1cc(-c2ccc3ncnc(Nc4cccc(Cl)c4F)c3c2)cnc1Cl] |
| Compound 5 | Compound 6 |
| 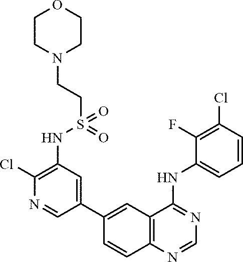 [SMILES: O=S(=O)(CCN1CCOCC1)Nc1cc(-c2ccc3ncnc(Nc4cccc(Cl)c4F)c3c2)cnc1Cl] | 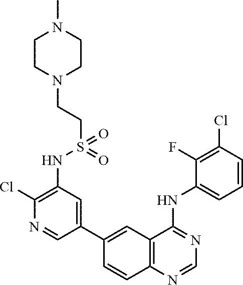 [SMILES: CN1CCN(CCS(=O)(=O)Nc2cc(-c3ccc4ncnc(Nc5cccc(Cl)c5F)c4c3)cnc2Cl)CC1] |
| Compound 7 | Compound 8 |
| 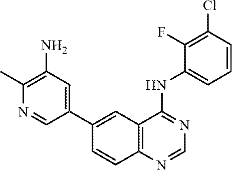 [SMILES: Cc1ncc(-c2ccc3ncnc(Nc4cccc(Cl)c4F)c3c2)cc1N] | 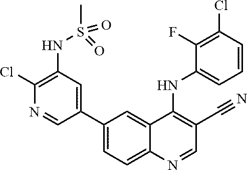 [SMILES: CS(=O)(=O)Nc1cc(-c2ccc3ncc(C#N)c(Nc4cccc(Cl)c4F)c3c2)cnc1Cl] |
| Compound 9 | Compound 10 |

### Pharmaceutical Compositions

The compounds described herein can be formulated into pharmaceutical compositions that further comprise a pharmaceutically acceptable carrier, diluent, adjuvant or vehicle. In one embodiment, the present invention provides a pharmaceutical composition comprising a compound of the invention described above, and a pharmaceutically acceptable carrier, diluent, adjuvant or vehicle. In one embodiment, the present invention is a pharmaceutical composition comprising an effective amount of a compound of the present invention or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

According to another embodiment, the invention provides a composition comprising a compound of this invention or a pharmaceutically acceptable salt thereof and a pharmaceutically acceptable carrier, adjuvant, or vehicle. Pharmaceutical compositions of this invention comprise a therapeutically effective amount of a compound of Formula I, wherein a “therapeutically effective amount” is an amount that is (a) effective to measurably modulate EGFR and/or PI3K in a biological sample or in a patient, or (b) effective in treating and/or ameliorating a disease or disorder that is mediated by EGFR and/or PI3K.

The term “patient,” as used herein, means an animal, preferably a mammal, and most preferably a human.

It also will be appreciated that certain of the compounds of the present invention can exist in free form for treatment, or where appropriate, as a pharmaceutically acceptable derivative (e.g., a salt) thereof. According to the present invention, a pharmaceutically acceptable derivative includes, but is not limited to, pharmaceutically acceptable prodrugs, salts, esters, salts of such esters, or any other adduct or derivative that upon administration to a patient in need is capable of providing, directly or indirectly, a compound as otherwise described herein, or a metabolite or residue thereof.

As used herein, the term “pharmaceutically acceptable salt” refers to those salts that are, within the scope of sound medical judgement, suitable for use in contact with the tissues of humans and lower animals without undue toxicity, irritation, allergic response and the like.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts include salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

A pharmaceutically acceptable carrier may contain inert ingredients that do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington’s Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, the use of such conventional carrier medium is contemplated to be within the scope of this invention. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful, uncomfortable, or risky. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

Some examples of materials that can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer’s solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents. Preservatives and antioxidants can also be present in the composition, according to the judgment of the formulator.

The compositions of the present invention may be administered orally, parenterally, by inhalation spray, topically, rectally, nasally, buccally, vaginally or via an implanted reservoir. As used herein, the term “parenteral” includes subcutaneous, intravenous, intramuscular, intra-articular, intra-synovial, intrasternal, intrathecal, intraocular, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation also may be a sterile injectable solution or suspension in a non-toxic parenterally-acceptable diluent or solvent, for example as a solution in 1,3-butanediol. Among the acceptable vehicles and solvents that may be employed are water, Ringer’s solution and isotonic sodium chloride solution. In addition, sterile, fixed oils are conventionally employed as a solvent or suspending medium.

For this purpose, any bland fixed oil may be employed including synthetic mono-or di-glycerides. Fatty acids, such as oleic acid and its glyceride derivatives, are useful in the preparation of injectables, as are natural pharmaceutically acceptable oils, such as olive oil or castor oil, especially in their polyoxyethylated versions. These oil solutions or suspensions also may contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers that are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents also may be added.

Alternatively, the pharmaceutically acceptable compositions of this invention may be administered in the form of suppositories for rectal or vaginal administration. These can be prepared by mixing the agent with a suitable non-irritating excipient that is solid at room temperature but liquid at rectal temperature and therefore will melt in the rectum or vaginal cavity to release the drug. Such materials include cocoa butter, polyethylene glycol or a suppository wax that is solid at ambient temperature but liquid at body temperature and therefore melt in the rectum or vaginal cavity and release the active compound.

The pharmaceutically acceptable compositions of this invention also may be administered topically, especially when the target of treatment includes areas or organs readily accessible by topical application, including diseases of the eye, skin, or lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches also may be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated, e.g., as micronized suspensions in isotonic, pH adjusted sterile saline or other aqueous solution, or, preferably, as solutions in isotonic, pH adjusted sterile saline or other aqueous solution, either with or without a preservative such as benzylalkonium chloride. Alternatively, for ophthalmic uses, the pharmaceutically acceptable compositions may be formulated in an ointment such as petrolatum. The pharmaceutically acceptable compositions of this invention also may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions also can include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation also may be a sterile injectable solution, suspension or emulsion in a nontoxic parenterally acceptable diluent or solvent, for example, as a solution in 1,3-butanediol. Among the acceptable vehicles and solvents that may be employed are water, Ringer’s solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid may be used in the preparation of injectables.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations also are prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form also may comprise buffering agents.

Solid compositions of a similar type also may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. Solid dosage forms optionally may contain opacifying agents. These solid dosage forms also can be of a composition such that they release the active ingredient(s) only, or preferentially, in a certain part of the intestinal tract, optionally, in a delayed manner. Examples of embedding compositions that can be used include polymeric substances and waxes. Solid compositions of a similar type also may be employed as fillers in soft and hard-filled gelatin capsules using such excipients as lactose or milk sugar as well as high molecular weight polethylene glycols and the like.

The active compounds also can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms also may comprise, as is normal practice, additional substances other than inert diluents, e.g., tableting lubricants and other tableting aids such a magnesium stearate and microcrystalline cellulose. In the case of capsules, tablets and pills, the dosage forms also may comprise buffering agents. They may optionally contain opacifying agents and also can be of a composition such that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops also are contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers also can be used to increase the flux of the compound across the skin. The rate can be controlled by either providing a rate controlling membrane or by dispersing the compound in a polymer matrix or gel.

The compounds of the invention preferably are formulated in dosage unit form for ease of administration and uniformity of dosage. As used herein, the phrase “dosage unit form” refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

The amount of the compounds of the present invention that may be combined with the carrier materials to produce a composition in a single dosage form will vary depending upon the host treated, the particular mode of administration, and other factors. Preferably, the compositions should be formulated so that a dosage of between 0.01 - 100 mg/kg body weight/day of the inhibitor can be administered to a patient receiving these compositions.

Depending upon the particular condition, or disease, to be treated or prevented, additional therapeutic agents, which are normally administered to treat or prevent that condition, also may be present in the compositions of this invention. As used herein, additional therapeutic agents that are normally administered to treat or prevent a particular disease, or condition, are known as “appropriate for the disease, or condition, being treated.”

Some embodiments of the present invention provide methods for administering an effective amount of a compound of the invention and at least one additional therapeutic agent (including, but not limited to, chemotherapeutic antineoplastics, apoptosis-modulating agents, antimicrobials, antivirals, antifungals, and anti-inflammatory agents) and/or therapeutic technique (e.g., surgical intervention, and/or radiotherapies). In a particular embodiment, the additional therapeutic agent(s) is an anticancer agent.

A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In certain embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present invention provide a compound of the invention and at least one anti-hyperproliferative or antineoplastic agent selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present invention include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

For example, chemotherapeutic agents or other anti-proliferative agents may be combined with the compounds of this invention to treat proliferative diseases and cancer. Examples of known chemotherapeutic agents include, but are not limited to, PI3K inhibitors (e.g., idelalisib and copanlisib), BCL-2 inhibitors (e.g., venetoclax), BTK inhibitors (e.g., ibrutinib and acalabrutinib), etoposide, CD20 antibodies (e.g., rituximab, ocrelizumab, obinutuzumab, ofatumumab, ibritumomab tiuxetan, tositumomab, and ublituximab), aletuzumab, bendamustine, cladribine, doxorubicin, chlorambucil, prednisone, midostaurin, lenalidomide, pomalidomide, checkpoint inhibitors (e.g., ipilimumab, nivolumab, pembolizumab, atezolizumab, avelumab, durvalumab), engineered cell therapy (e.g., CAR-T therapy -Kymriah®, Yescarta®), Gleevec™, adriamycin, dexamethasone, vincristine, cyclophosphamide, fluorouracil, topotecan, taxol, interferons, and platinum derivatives.

And, in some instances, radiation therapy is administered during the treatment course wherein a compound of the present invention (or a pharmaceutically acceptable salt thereof) is administered to a patient in need thereof.

Other examples of agents with which the compounds of this invention also may be combined include, chemotherapeutic agents such as Abecma (Idecabtagene Vicleucel), Abemaciclib, Abiraterone Acetate, Abraxane (Paclitaxel Albumin-stabilized Nanoparticle Formulation), ABVD, ABVE, ABVE-PC, AC, Acalabrutinib, AC-T, Actemra (Tocilizumab), Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alpelisib, Alunbrig (Brigatinib), Ameluz (Aminolevulinic Acid Hydrochloride), Amifostine, Aminolevulinic Acid Hydrochloride, Anastrozole, Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Asparlas (Calaspargase Pegol-mknl), Atezolizumab, Avapritinib, Avastin (Bevacizumab), Avelumab, Axicabtagene Ciloleucel, Axitinib, Ayvakit (Avapritinib), Azacitidine, Azedra (Iobenguane I 131), Balversa (Erdafitinib), Bavencio (Avelumab), BEACOPP, Belantamab Mafodotin-blmf, Beleodaq (Belinostat), Belinostat, Bendamustine Hydrochloride, Bendeka (Bendamustine Hydrochloride), BEP, Besponsa (Inotuzumab Ozogamicin), Bevacizumab, Bexarotene, Bicalutamide, BiCNU (Carmustine), Binimetinib, Blenrep (Belantamab Mafodotin-blmf), Bleomycin Sulfate, Blinatumomab, Blincyto (Blinatumomab), Bortezomib, Bosulif (Bosutinib), Bosutinib, Braftovi (Encorafenib), Brentuximab Vedotin, Brexucabtagene Autoleucel, Breyanzi (Lisocabtagene Maraleucel), Brigatinib, Brukinsa (Zanubrutinib), BuMel, Busulfan, Busulfex (Busulfan), Cabazitaxel, Cablivi (Caplacizumab-yhdp), Cabometyx (Cabozantinib-S-Malate), Cabozantinib-S-Malate, CAF, Calaspargase Pegol-mknl, Calquence (Acalabrutinib), Campath (Alemtuzumab), Camptosar (Irinotecan Hydrochloride), Capecitabine, Caplacizumab-yhdp, Capmatinib Hydrochloride, CAPOX, Carac (Fluorouracil-Topical), Carboplatin, CARBOPLATIN-TAXOL, Carfilzomib, Carmustine, Carmustine Implant, Casodex (Bicalutamide), CEM, Cemiplimab-rwlc, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar (Clofarabine), CMF, Cobimetinib Fumarate, Cometriq (Cabozantinib-S-Malate), Copanlisib Hydrochloride, COPDAC, Copiktra (Duvelisib), COPP, COPP-ABV, Cosmegen (Dactinomycin), Cotellic (Cobimetinib Fumarate), Crizotinib, CVP, Cyclophosphamide, Cyramza (Ramucirumab), Cytarabine, Dabrafenib Mesylate, Dacarbazine, Dacogen (Decitabine), Dacomitinib, Dactinomycin, Danyelza (Naxitamab-gqgk), Daratumumab, Daratumumab and Hyaluronidase-fihj, Darbepoetin Alfa, Darolutamide, Darzalex (Daratumumab), Darzalex Faspro (Daratumumab and Hyaluronidase-fihj), Dasatinib, Daunorubicin Hydrochloride, Daunorubicin Hydrochloride and Cytarabine Liposome, Daurismo (Glasdegib Maleate), Decitabine, Decitabine and Cedazuridine, Defibrotide Sodium, Defitelio (Defibrotide Sodium), Degarelix, Denileukin Diftitox, Denosumab, Dexamethasone, Dexrazoxane Hydrochloride, Dinutuximab, Docetaxel, Dostarlimab-gxly, Doxil (Doxorubicin Hydrochloride Liposome), Doxorubicin Hydrochloride, Doxorubicin Hydrochloride Liposome, Durvalumab, Duvelisib, Efudex (Fluorouracil-Topical), Eligard (Leuprolide Acetate), Elitek (Rasburicase), Ellence (Epirubicin Hydrochloride), Elotuzumab, Eloxatin (Oxaliplatin), Eltrombopag Olamine, Elzonris (Tagraxofusp-erzs), Emapalumab-lzsg, Emend (Aprepitant), Empliciti (Elotuzumab), Enasidenib Mesylate, Encorafenib, Enfortumab Vedotin-ejfv, Enhertu (Fam-Trastuzumab Deruxtecan-nxki), Entrectinib, Enzalutamide, Epirubicin Hydrochloride, EPOCH, Epoetin Alfa, Epogen (Epoetin Alfa), Erbitux (Cetuximab), Erdafitinib, Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos (Etoposide Phosphate), Etoposide, Etoposide Phosphate, Everolimus, Evista (Raloxifene Hydrochloride), Evomela (Melphalan Hydrochloride), Exemestane, 5-FU (Fluorouracil Injection), 5-FU (Fluorouracil-Topical), Fam-Trastuzumab Deruxtecan-nxki, Fareston (Toremifene), Farydak (Panobinostat Lactate), Faslodex (Fulvestrant), FEC, Fedratinib Hydrochloride, Femara (Letrozole), Filgrastim, Firmagon (Degarelix), Fludarabine Phosphate, Fluoroplex (Fluorouracil-Topical), Fluorouracil Injection, Fluorouracil—Topical, Flutamide, FOLFIRI, FOLFIRI-BEVACIZUMAB, FOLFIRI-CETUXIMAB, FOLFIRINOX, FOLFOX, Folotyn (Pralatrexate), Fostamatinib Disodium, Fotivda (Tivozanib Hydrochloride), Fulphila (Pegfilgrastim), FU-LV, Fulvestrant, Gamifant (Emapalumab-lzsg), Gardasil (Recombinant HPV Quadrivalent Vaccine), Gardasil 9 (Recombinant HPV Nonavalent Vaccine), Gavreto (Pralsetinib), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gilteritinib Fumarate, Glasdegib Maleate, Gleevec (Imatinib Mesylate), Gliadel Wafer (Carmustine Implant), Glucarpidase, Goserelin Acetate, Granisetron, Granisetron Hydrochloride, Granix (Filgrastim), Halaven (Eribulin Mesylate), Hemangeol (Propranolol Hydrochloride), Herceptin Hylecta (Trastuzumab and Hyaluronidase-oysk), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin PFS (Idarubicin Hydrochloride), Idarubicin Hydrochloride, Idecabtagene Vicleucel , Idelalisib, Idhifa (Enasidenib Mesylate), Ifex (Ifosfamide), Ifosfamide, IL-2 (Aldesleukin), Imatinib Mesylate, Imbruvica (Ibrutinib), Imfinzi (Durvalumab), Imiquimod, Imlygic (Talimogene Laherparepvec), Infugem (Gemcitabine Hydrochloride), Inlyta (Axitinib), Inotuzumab Ozogamicin, Inqovi (Decitabine and Cedazuridine), Inrebic (Fedratinib Hydrochloride), Interferon Alfa-2b, Recombinant, Interleukin-2 (Aldesleukin), Intron A (Recombinant Interferon Alfa-2b), Iobenguane 1131, Ipilimumab, Iressa (Gefitinib), Irinotecan Hydrochloride, Irinotecan Hydrochloride Liposome, Isatuximab-irfc, Istodax (Romidepsin), Ivosidenib, Ixabepilone, Ixazomib Citrate, Ixempra (Ixabepilone), Jakafi (Ruxolitinib Phosphate), JEB, Jelmyto (Mitomycin), Jemperli (Dostarlimab-gxly), Jevtana (Cabazitaxel), Kadcyla (Ado-Trastuzumab Emtansine), Kepivance (Palifermin), Keytruda (Pembrolizumab), Kisqali (Ribociclib), Koselugo (Selumetinib Sulfate), Kymriah (Tisagenlecleucel), Kyprolis (Carfilzomib), Lanreotide Acetate, Lapatinib Ditosylate, Larotrectinib Sulfate, Lenalidomide, Lenvatinib Mesylate, Lenvima (Lenvatinib Mesylate), Letrozole, Leucovorin Calcium, Leukeran (Chlorambucil), Leuprolide Acetate, Levulan Kerastik (Aminolevulinic Acid Hydrochloride), Libtayo (Cemiplimab-rwlc), Lisocabtagene Maraleucel, Lomustine, Loncastuximab Tesirine-lpyl, Lonsurf (Trifluridine and Tipiracil Hydrochloride), Lorbrena (Lorlatinib), Lorlatinib, Lumakras (Sotorasib), Lumoxiti (Moxetumomab Pasudotox-tdfk), Lupron Depot (Leuprolide Acetate), Lurbinectedin, Luspatercept-aamt, Lutathera (Lutetium Lu 177-Dotatate), Lutetium (Lu 177-Dotatate), Lynparza (Olaparib), Margenza (Margetuximab-cmkb), Margetuximab-cmkb, Marqibo (Vincristine Sulfate Liposome), Matulane (Procarbazine Hydrochloride), Mechlorethamine Hydrochloride, Megestrol Acetate, Mekinist (Trametinib Dimethyl Sulfoxide), Mektovi (Binimetinib), Melphalan, Melphalan Flufenamide Hydrochloride, Melphalan Hydrochloride, Mercaptopurine, Mesna, Mesnex (Mesna), Methotrexate Sodium, Methylnaltrexone Bromide, Midostaurin, Mitomycin , Mitoxantrone Hydrochloride, Mogamulizumab-kpkc, Monjuvi (Tafasitamab-cxix), Moxetumomab Pasudotox-tdfk, Mozobil (Plerixafor), MVAC, Mvasi (Bevacizumab), Myleran (Busulfan), Mylotarg (Gemtuzumab Ozogamicin), Nanoparticle Paclitaxel (Paclitaxel Albumin-stabilized Nanoparticle Formulation), Naxitamab-gqgk, Necitumumab, Nelarabine, Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivestym (Filgrastim), Nivolumab, Nplate (Romiplostim), Nubeqa (Darolutamide), Nyvepria (Pegfilgrastim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Onureg (Azacitidine), Opdivo (Nivolumab), OPPA, Orgovyx (Relugolix), Osimertinib Mesylate, Oxaliplatin, Paclitaxel, Paclitaxel Albumin-stabilized Nanoparticle Formulation, PAD, Padcev (Enfortumab Vedotin-ejfv), Palbociclib, Palifermin, Palonosetron Hydrochloride, Palonosetron Hydrochloride and Netupitant, Pamidronate Disodium, Panitumumab, Panobinostat Lactate, Pazopanib Hydrochloride, PCV, PEB, Pegaspargase, Pegfilgrastim, Peginterferon Alfa-2b, PEG-Intron (Peginterferon Alfa-2b), Pemazyre (Pemigatinib), Pembrolizumab, Pemetrexed Disodium, Pemigatinib, Peptaxto (Melphalan Flufenamide Hydrochloride), Perjeta (Pertuzumab), Pertuzumab, Pertuzumab, Trastuzumab, and Hyaluronidase-zzxf, Pexidartinib Hydrochloride, Phesgo (Pertuzumab, Trastuzumab, and Hyaluronidase-zzxf), Piqray (Alpelisib), Plerixafor, Polatuzumab Vedotin-piiq, Polivy (Polatuzumab Vedotin-piiq), Pomalidomide, Pomalyst (Pomalidomide), Ponatinib Hydrochloride, Portrazza (Necitumumab), Poteligeo (Mogamulizumab-kpkc), Pralatrexate, Pralsetinib, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Qinlock (Ripretinib), Radium 223 Dichloride, Raloxifene Hydrochloride, Ramucirumab, Rasburicase, Ravulizumab-cwvz, Reblozyl (Luspatercept-aamt), R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), Relugolix, R-EPOCH, Retacrit (Epoetin Alfa), Retevmo (Selpercatinib), Revlimid (Lenalidomide), Ribociclib, R-ICE, Ripretinib, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rozlytrek (Entrectinib), Rubidomycin (Daunorubicin Hydrochloride), Rubraca (Rucaparib Camsylate), Rucaparib Camsylate, Ruxolitinib Phosphate, Rydapt (Midostaurin), Sacituzumab Govitecan-hziy, Sancuso (Granisetron), Sarclisa (Isatuximab-irfc), Sclerosol Intrapleural Aerosol (Talc), Selinexor, Selpercatinib, Selumetinib Sulfate, Siltuximab, Sipuleucel-T, Soltamox (Tamoxifen Citrate), Somatuline Depot (Lanreotide Acetate), Sonidegib, Sorafenib Tosylate, Sotorasib, Sprycel (Dasatinib), STANFORD V, Sterile Talc Powder (Talc), Steritalc (Talc), Stivarga (Regorafenib), Sunitinib Malate, Sustol (Granisetron), Sutent (Sunitinib Malate), Sylatron (Peginterferon Alfa-2b), Sylvant (Siltuximab), Synribo (Omacetaxine Mepesuccinate), Tabloid (Thioguanine), Tabrecta (Capmatinib Hydrochloride), TAC, Tafasitamab-cxix, Tafinlar (Dabrafenib Mesylate), Tagraxofusp-erzs, Tagrisso (Osimertinib Mesylate), Talazoparib Tosylate, Talc, Talimogene Laherparepvec, Talzenna (Talazoparib Tosylate), Tamoxifen Citrate, Tarceva (Erlotinib Hydrochloride), Targretin (Bexarotene), Tasigna (Nilotinib), Tavalisse (Fostamatinib Disodium), Taxotere (Docetaxel), Tazemetostat Hydrobromide, Tazverik (Tazemetostat Hydrobromide), Tecartus (Brexucabtagene Autoleucel), Tecentriq (Atezolizumab), Temodar (Temozolomide), Temozolomide, Temsirolimus, Tepadina (Thiotepa), Tepmetko (Tepotinib Hydrochloride), Tepotinib Hydrochloride, Thalidomide, Thalomid (Thalidomide), Thioguanine, Thiotepa, Tibsovo (Ivosidenib), Tisagenlecleucel, Tivozanib Hydrochloride, Tocilizumab, Tolak (Fluorouracil—Topical), Topotecan Hydrochloride, Toremifene, Torisel (Temsirolimus), Totect (Dexrazoxane Hydrochloride), TPF, Trabectedin, Trametinib Dimethyl Sulfoxide, Trastuzumab, Trastuzumab and Hyaluronidase-oysk, Treanda (Bendamustine Hydrochloride), Trexall (Methotrexate Sodium), Trifluridine and Tipiracil Hydrochloride, Trisenox (Arsenic Trioxide), Trodelvy (Sacituzumab Govitecan-hziy), Truxima (Rituximab), Tucatinib, Tukysa (Tucatinib), Turalio (Pexidartinib Hydrochloride), Tykerb (Lapatinib Ditosylate), Ukoniq (Umbralisib Tosylate), Ultomiris (Ravulizumab-cwvz), Umbralisib Tosylate, Undencyca (Pegfilgrastim), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velcade (Bortezomib), Vemurafenib, Venclexta (Venetoclax), Venetoclax, Verzenio (Abemaciclib), Vidaza (Azacitidine), Vinblastine Sulfate, Vincristine Sulfate, Vincristine Sulfate Liposome, Vinorelbine Tartrate, VIP, Vismodegib, Vistogard (Uridine Triacetate), Vitrakvi (Larotrectinib Sulfate), Vizimpro (Dacomitinib), Voraxaze (Glucarpidase), Vorinostat, Votrient (Pazopanib Hydrochloride), Vyxeos (Daunorubicin Hydrochloride and Cytarabine Liposome), Xalkori (Crizotinib), Xatmep (Methotrexate Sodium), Xeloda (Capecitabine), XELIRI, XELOX, Xgeva (Denosumab), Xofigo (Radium 223 Dichloride), Xospata (Gilteritinib Fumarate), Xpovio (Selinexor), Xtandi (Enzalutamide), Yervoy (Ipilimumab), Yescarta (Axicabtagene Ciloleucel), Yondelis (Trabectedin), Yonsa (Abiraterone Acetate), Zaltrap (Ziv-Aflibercept), Zanubrutinib , Zarxio (Filgrastim), Zejula (Niraparib Tosylate Monohydrate), Zelboraf (Vemurafenib), Zepzelca (Lurbinectedin), Zevalin (Ibritumomab Tiuxetan), Ziextenzo (Pegfilgrastim), Zinecard (Dexrazoxane Hydrochloride), Zirabev (Bevcizumab), Ziv-Aflibercept, Zofran (Ondansetron Hydrochloride), Zoladex (Goserelin Acetate), Zoledronic Acid, Zolinza (Vorinostat), Zometa (Zoledronic Acid), Zyclara (Imiquimod), Zydelig (Idelalisib), Zykadia (Ceritinib), Zynlonta (Loncastuximab Tesirine-lpyl), and Zytiga (Abiraterone Acetate).

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflomithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician’s Desk Reference and to Goodman and Gilman’s “Pharmaceutical Basis of Therapeutics” tenth edition, Eds. Hardman et al., 2002.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

### Methods of Treatment

The compounds of the invention are modulators (e.g., inhibitors) of the activity or function of proteins of the phosphoinositide 3′ OH kinase family (PIK3) (e.g., PIK3Cα, PIK3δ, PIK3β, PIK3Cγ, PI3Kα) and modulation (e.g., inhibition) of the activity or function of proteins of the epidermal growth factor EGFR family (e.g., ERBB receptor tyrosine kinase family (e.g., ERBB1, ERBB2, ERBB4, ERBB1)).

PI3K is negatively regulated by phosphatase and tensin homolog (PTEN) (see, e.g., Hamada K, et al., 2005 Genes Dev 19 (17): 2054-65). Numerous studies have shown a link between PIK3CA mutation/PTEN loss and EGFR targeted resistance leading to poor overall survival (see, e.g., Atreya CE, Sangale Z, Xu N, et al. Cancer Med. 2013;2: 496-506; Sawai H, et al., BMC Gastroenterol. 2008;8: 56; Bethune G, et al., J Thorac Dis. 2010;2: 48-51; Spano JP, et al., Ann Oncol. 2005;16: 189-194; Heimberger AB, et al., J Transl Med. 2005;3: 38). The quinazoline compounds and quinoline compounds synthesized during the course of developing embodiments for the present invention were designed based on a central hypothesis that dual targeting of EGFR and PIK3CA would be efficacious in patients with colorectal cancer that are EGFR positive and are either PIK3CA mutated or null PTEN expressers (see, e.g., Psyrri A, et al., Am Soc Clin Oncol Educ Book. 2013: 246-255; Lui VW, et al., Cancer Discov. 2013;3: 761-769; Jin G, et al., Lung Cancer. 2010;69: 279-283; Buck E, et al., Mol Cancer Ther. 2006;5: 2676-2684; Fan QW, et al., Cancer Res. 2007;67: 7960-7965; Gadgeel SM, et al., Clin Lung Cancer. 2013; 14: 322-332.

As such, the present invention relates to a new class of small-molecules having a quinazoline structure or quinoline structure which function as dual inhibitors of EGFR protein and PI3K protein, and their use as therapeutics for the treatment of conditions characterized by aberrant EGFR and PI3K expression (e.g., cancer and other diseases (e.g., autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection, lung injuries, etc)). Indeed, through targeting both EGFR and PI3K, the compounds of the present invention are useful in treating subjects with EGFR positive colorectal cancer that harbor an activating mutation in PI3Kα or are PTEN null.

Accordingly, the present invention contemplates that exposure of patients (e.g., humans) suffering from a condition characterized by aberrant EGFR protein activity (e.g., ERBB1) and PI3K protein activity (e.g., PI3Kα) (e.g., cancer (e.g., and/or cancer related disorders)) to therapeutically effective amounts of drug(s) having a quinazoline structure (e.g., small molecules having a quinazoline structure) or a quinoline structures (e.g., small molecules having a quinoline structure) that inhibit the activity of both EGFR and PI3K will inhibit the growth of cells characterized by aberrant EGFR and PI3K protein expression (e.g., colorectal cancer cells having aberrant EGFR and PI3K protein expression) and/or render such cells as a population more susceptible to the cell death-inducing activity of additional therapeutic drugs (e.g., cancer therapeutic drugs or radiation therapies). The present invention contemplates that inhibitors of both EGFR and PI3K satisfy an unmet need for the treatment of multiple conditions characterized with aberrant EGFR and PI3K activity (e.g., cancer), either when administered as monotherapy to induce cell growth inhibition, apoptosis and/or cell cycle arrest in such cells (e.g., cancer cells), or when administered in a temporal relationship with additional agent(s), such as other cell death-inducing or cell cycle disrupting therapeutic drugs (e.g., cancer therapeutic drugs or radiation therapies) (combination therapies), so as to render a greater proportion of the cells (e.g., cancer cells) or supportive cells susceptible to executing the apoptosis program compared to the corresponding proportion of cells in a patient treated only with the therapeutic drug or radiation therapy alone.

In certain embodiments of the invention wherein the condition being treated is cancer characterized with aberrant EGFR protein activity (e.g., ERBB1) and PI3K protein activity (e.g., PI3Kα) (e.g., colorectal cancer), combination treatment of patients with a therapeutically effective amount of a compound of the present invention and a course of an anticancer agent produces a greater tumor response and clinical benefit in such patients compared to those treated with the compound or anticancer drugs/radiation alone. Since the doses for all approved anticancer drugs and radiation treatments are known, the present invention contemplates the various combinations of them with the present compounds.

As noted, the Applicants have found that certain quinazoline compounds and quinoline compounds function as inhibitors of both EGFR and PI3K, and serve as therapeutics for the treatment of cancer and other diseases. Thus, the present invention relates to quinazoline compounds and quinoline compounds useful for inhibiting EGFR and PI3K activity (e.g., thereby facilitating cell apoptosis), and increasing the sensitivity of cells to inducers of apoptosis and/or cell cycle arrest. Certain quinazoline compounds and quinoline compounds of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

The invention also provides the use of compounds to induce cell cycle arrest and/or apoptosis in cells characterized with aberrant EGFR protein activity (e.g., ERBB1) and PI3K protein activity (e.g., PI3Kα). The invention also relates to the use of compounds for sensitizing cells to additional agent(s), such as inducers of apoptosis and/or cell cycle arrest, and chemoprotection of normal cells through the induction of cell cycle arrest prior to treatment with chemotherapeutic agents.

The compounds of the invention are useful for the treatment, amelioration, or prevention of disorders, such as those responsive to induction of apoptotic cell death, e.g., disorders characterized by dysregulation of apoptosis, including hyperproliferative diseases such as cancer characterized with cells aberrant EGFR protein activity (e.g., ERBB1) and PI3K protein activity (e.g., PI3Kα) (e.g., colorectal cancer). In certain embodiments, the compounds can be used to treat, ameliorate, or prevent such types of cancer (e.g., colorectal cancer) that is characterized by resistance to cancer therapies (e.g., those cancer cells which are chemoresistant, radiation resistant, hormone resistant, and the like). In certain embodiments, the cancer is colorectal cancer, head & neck cancer, glioblastoma multiform, and/or non-small cell lung cancer (NSCLC). In other embodiments, the compounds can be used to treat other characterized by aberrant expression of EGFR and PI3K proteins (e.g., autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection, lung injuries, etc).

The invention also provides pharmaceutical compositions comprising the compounds of the invention in a pharmaceutically acceptable carrier.

The invention also provides kits comprising a compound of the invention and instructions for administering the compound to a patient. The kits may optionally contain other therapeutic agents, e.g., anticancer agents or apoptosis-modulating agents.

Moreover, the present invention provides methods for simultaneously inhibiting both EGFR protein activity and PI3K protein activity in cells through exposing such cells to one or more of the quinazoline or quinoline compounds of the present invention.

In spite of compelling evidence for PI3K/AKT pathway activation leading to resistance to EGFR targeting agents, only recently have researchers sought to combine EGFR targeting agents with PI3K/AKT/MTOR pathway inhibitors both pre-clinically and clinically. For example, Buck et al demonstrated that the mTOR inhibitor rapamycin synergizes with the EGFR inhibitor erlotinib in several cell lines that were resistant to erlotinib treatment alone (e.g., Ratushny V, et al., Cell Signal. 2009;21: 1255-1268). However, the full potential of this synergistic combination was not achieved because rapamycin induces phosphorylation of AKT resulting in pathway reactivation (e.g., Ratushny V, et al., Cell Signal. 2009;21: 1255-1268). Others have explored dual inhibition of EGFR and PI3K/AKT pathways in several cell lines and cancer histotypes, providing further support for this combination treatment strategy (see, e.g., Eichhorn PJ, et al., Cancer Res. 2008;68: 9221-9230). The compounds of the present invention overcame such limitations and represent dual potency inhibitors of both EGFR protein activity (e.g., ERBB1) and PI3K protein activity (e.g., PI3Kα). Specifically, utilizing x-ray crystal structure and structure-activity relationships gleaned from known PI3K and EGFR inhibiting agents, such experiments resulted in the identification of “active cores” for PI3K inhibiting agents facilitating high inhibitory activity against PI3K, and the identification of “active cores” for EGFR inhibiting agents facilitating high inhibitory activity against EGFR, respectively (see, Example I). The quinazoline and quinoline compounds of the present invention were accordingly synthesized to target the “active cores” for PI3K and the “active cores” for EGFR, thereby rendering such compounds as having “dual potency” against EGFR protein activity (e.g., ERBB1) and PI3K protein activity (e.g., PI3Ka).

Accordingly, the present invention relates to compounds which function as inhibitors of EGFR protein activity (e.g., ERBB1) and PI3K protein activity (e.g., PI3Kα). By inhibiting the activity of EGFR protein activity (e.g., ERBB1) and PI3K protein activity (e.g., PI3Kα), these compounds sensitize cells to inducers of apoptosis and/or cell cycle arrest and, in some instances, themselves induce apoptosis and/or cell cycle arrest. Therefore, the invention relates to methods of sensitizing cells to inducers of apoptosis and/or cell cycle arrest and to methods of inducing apoptosis and/or cell cycle arrest in cells, comprising contacting the cells with a compound of the invention alone or in combination with additional agent(s), e.g., an inducer of apoptosis or a cell cycle disrupter.

The invention further relates to methods of treating, ameliorating, or preventing conditions in a patient characterized with cells having aberrant EGFR protein activity (e.g., ERBB1) and PI3K protein activity (e.g., PI3Kα), such as those conditions that are responsive to induction of apoptosis, comprising administering to the patient a compound of the invention and additional agent(s), e.g., an inducer of apoptosis. Such disorders include those characterized by a dysregulation of apoptosis and those characterized by the proliferation of cells having aberrant EGFR protein activity (e.g., ERBB1) and PI3K protein activity (e.g., PI3Kα) (e.g., colorectal cancer). Indeed, through targeting both EGFR and PI3K, the compounds of the present invention are useful in treating subjects with EGFR positive colorectal cancer that harbor an activating mutation in PI3Kα or are PTEN null.

An important aspect of the present invention is that compounds of the invention induce cell cycle arrest and/or apoptosis and also potentiate the induction of cell cycle arrest and/or apoptosis either alone or in response to additional apoptosis induction signals. Therefore, it is contemplated that these compounds sensitize cells to induction of cell cycle arrest and/or apoptosis, including cells that are resistant to such inducing stimuli. The EGFR and PI3K inhibitors of the present invention (e.g., quinazoline compounds) (e.g., quinoline compounds) can be used to induce apoptosis in any disorder that can be treated, ameliorated, or prevented by the induction of apoptosis.

In some embodiments, the compositions and methods of the present invention are used to treat diseased cells, tissues, organs, or pathological conditions and/or disease states in a patient (e.g., a mammalia patient including, but not limited to, humans and veterinary animals). In this regard, various diseases and pathologies are amenable to treatment or prophylaxis using the present methods and compositions. A non-limiting exemplary list of these diseases and conditions includes, but is not limited to, colorectal cancer, non-small cell lung carcinoma, head or neck carcinoma, glioblastoma multiform cancer, pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, , breast carcinoma, ovarian carcinoma, lung carcinoma, small-cell lung carcinoma, Wilms’ tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi’s sarcoma, polycythemia vera, essential thrombocytosis, Hodgkin’s disease, non-Hodgkin’s lymphoma, soft-tissue sarcoma, osteogenic sarcoma, primary macroglobulinemia, and retinoblastoma, and the like, T and B cell mediated autoimmune diseases; inflammatory diseases; infections; hyperproliferative diseases; AIDS; degenerative conditions, vascular diseases, and the like. In some embodiments, the cancer cells being treated are metastatic. In other embodiments, the cancer cells being treated are resistant to anticancer agents.

In other embodiments, the disorder is any disorder having cells having aberrant EGFR protein activity (e.g., ERBB1) and PI3K protein activity (e.g., PI3Kα) (e.g., autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection, lung injuries, etc)).

The present invention provides methods for administering a compound of the invention with radiation therapy. The invention is not limited by the types, amounts, or delivery and administration systems used to deliver the therapeutic dose of radiation to a patient. For example, the patient may receive photon radiotherapy, particle beam radiation therapy, other types of radiotherapies, and combinations thereof. In some embodiments, the radiation is delivered to the patient using a linear accelerator. In still other embodiments, the radiation is delivered using a gamma knife.

The source of radiation can be external or internal to the patient. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by patients. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The patient may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to a patient, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to a patient is about .01 Gray (Gy) to about 100 Gy. In another embodiment, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the patient to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the patient’s responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present invention.

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In some embodiments of the present invention, a compound of the invention and one or more therapeutic agents or anticancer agents are administered to a patient under one or more of the following conditions: at different periodicities, at different durations, at different concentrations, by different administration routes, etc. In some embodiments, the compound is administered prior to the therapeutic or anticancer agent, e.g., 0.5, 1, 2, 3, 4, 5, 10, 12, or 18 hours, 1, 2, 3, 4, 5, or 6 days, or 1, 2, 3, or 4 weeks prior to the administration of the therapeutic or anticancer agent. In some embodiments, the compound is administered after the therapeutic or anticancer agent, e.g., 0.5, 1, 2, 3, 4, 5, 10, 12, or 18 hours, 1, 2, 3, 4, 5, or 6 days, or 1, 2, 3, or 4 weeks after the administration of the anticancer agent. In some embodiments, the compound and the therapeutic or anticancer agent are administered concurrently but on different schedules, e.g., the compound is administered daily while the therapeutic or anticancer agent is administered once a week, once every two weeks, once every three weeks, or once every four weeks. In other embodiments, the compound is administered once a week while the therapeutic or anticancer agent is administered daily, once a week, once every two weeks, once every three weeks, or once every four weeks.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary patients (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

### General Synthetic Procedures

Additional embodiments are disclosed in further detail in the following general synthetic procedures and specific synthetic examples, which are not in any way intended to limit the scope of the claims.

The exemplary compounds of general Formula A can be synthesized according to Scheme 1 below.

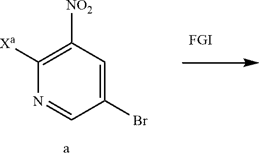

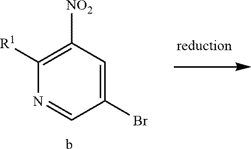

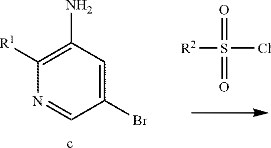

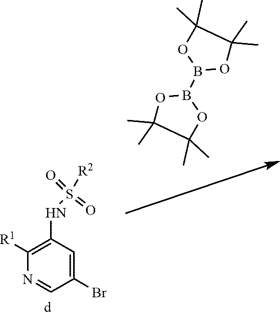

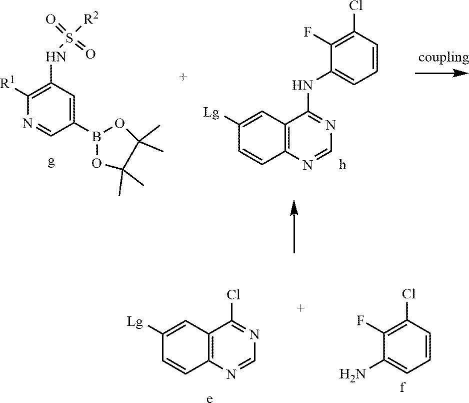

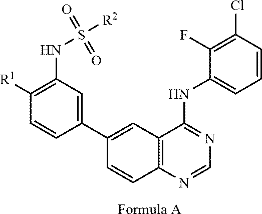

Compounds of formula b can be synthesized from a compound of formula a, wherein X^{a} is a leaving group, such as chloride, or other chemical moiety susceptible to conversion to an appropriate R¹ group, wherein R¹ is defined herein. Compounds of formula b can then be converted to compounds of formula c under reducing conditions, such as an Fe⁰ reagent in the presence of ammonium chloride. An L-R² moiety, wherein L is a sulfone and R² is defined herein, can be attached to the amine substituent of the compound of formula c to produce a compound of formula d by reacting, for example, with the appropriate sulfonyl chloride. The compound of formula d can then be converted to a compound of formula g by reacting with a boronating reagent, such as bis(pinacolato)diboron under coupling conditions. Exemplary coupling conditions for this transformation utilize a palladium catalyst, such as Pd(dppf)Cl₂ in the presence of a base such as potassium acetate. Compounds of formula h can be synthesized from the coupling of a compound of formula e, wherein Lg is a leaving group, and a compound of formula f under coupling conditions. Exemplary coupling conditions for this transformation also utilize a palladium catalyst, such as Pd(dppf)Cl₂ in the presence of a base such as cesium carbonate and a solvent, such as a DMF and water mixture. Reacting a compound of formula g with a compound of formula h under, for example Suzuki coupling conditions using a Pd⁰ catalyst such as Pd(PPh₃)₄ and a base such as potassium tert-butoxide or potassium carbonate in a suitable solvent, can provide a compound of Formula A.

### Examples

### Example 1: Synthesis of N-[5-[4-(3-chloro-2-fluoro-anilino) Quinazolin-6-yl]-2-methyl-3-pyridyl] Methanesulfonamide (Compound 1)

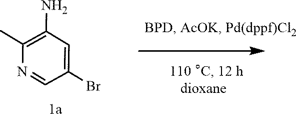

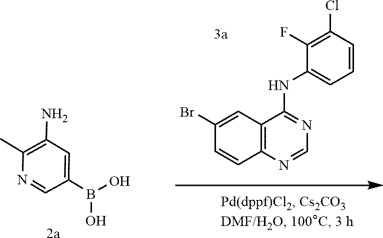

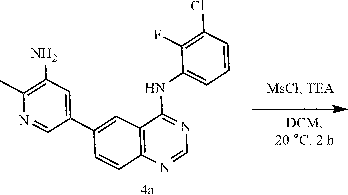

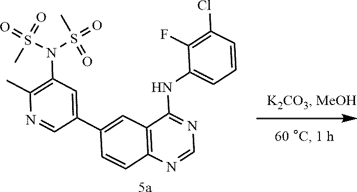

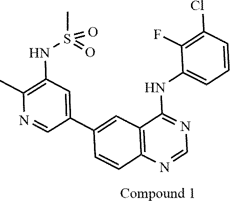

### Step 1: Synthesis of (5-amino-6-methyl-3-pyridyl)boronic acid (2a)

To a solution of 5-bromo-2-methyl-pyridin-3-amine (1a; 500 mg, 2.67 mmol, 1 eq) in dioxane (10 mL) was added AcOK (787.08 mg, 8.02 mmol, 3 eq), Pd(dppf)Cl₂ (195.60 mg, 267.33 µmol, 0.1 eq) and BPD (678.84 mg, 2.67 mmol, 1 eq), the mixture was purged with Ar for 3 times, the reaction was stirred at 110° C. for 12 h. LCMS showed starting material was consumed completely and the MS of desired product was detected. The reaction was filtered, and the filtrate was concentrated in vacuum. The crude product was purified by prep-HPLC (column: Phenomenex luna C18 80*40 mm*3 µm; mobile phase: [water (0.04%HCl)-ACN]; B%: 1%-10%,5 min). The product was obtained as a yellow solid (300 mg, 1.59 mmol, 59.56% yield, HCl). MS (M + H)⁺ =163.3

### Step 2: Synthesis of 6-(5-amino-6-methyl-3-pyridyl)-N-(3-chloro-2-fluoro-phenyl) quinazolin-4-amine (4a)

To a solution of Compound 2a (80 mg, 526.46 µmol, 1 eq) in DMF (0.5 mL) and H₂O (0.1 mL) was added Cs₂CO₃ (514.59 mg, 1.58 mmol, 3 eq), 6-bromo-N-(3-chloro-2-fluorophenyl)quinazolin-4-amine (3a; 185.62 mg, 526.46 µmol, 1 eq) and Pd(dppf)Cl₂ (38.52 mg, 52.65 µmol, 0.1 eq), the mixture was bubbled with N₂, the reaction was stirred at 100° C. for 3 h. LCMS showed starting material was consumed completely and the MS of desired product was detected. The mixture was extracted with ethyl acetate (2 × 10 mL). The combined organics were washed with brine (10 mL), dried over Na₂SO₄, filtered, and concentrated under reduced pressure to give the desired product (120 mg, 230.64 µmol, 43.81% yield, 73% purity based on TLC (DCM: MeOH = 10: 1, Rf = 0.58)) as a black solid. ¹H NMR (400 MHz, DMSO-d₆) δ ppm 10.17 (br s, 1 H), 8.74 (br s, 1 H), 8.51 (br s, 1 H), 8.18 (s, 1 H), 8.10 (br d, J=9.26 Hz, 1 H), 7.85 - 7.96 (m, 1 H), 7.48 - 7.56 (m, 2 H), 7.33 (d, J=1.88 Hz, 1 H), 2.39 (m, 3 H). MS (M + H)⁺ = 380.2

### Step 3: Synthesis N-[5-[4-(3-chloro-2-fluoro-anilino) quinazolin-6-yl] -2-methyl-3-pyridyl] -N-methylsulfonyl-methanesulfonamide (5a)

To a stirred solution of Compound 4a (100 mg, 263.28 µmol, 1 eq) in DCM (3 mL) was added dropwise TEA (106.57 mg, 1.05 mmol, 146.58 µL, 4 eq) and MsCl (150.80 mg, 1.32 mmol, 101.89 µL, 5 eq) at 0° C. under N₂. Then the mixture was stirred at 0° C. for 2 h. LCMS showed starting material was consumed completely and the MS of desired product was detected. The reaction mixture was poured into MeOH (10 ml), the pH of the mixture was adjust to 8~9, and the reaction was concentrated in vacuum. The desired product (150 mg, crude) was obtained as a brown oil. MS (M + H)⁺ = 536.0

### Step 4: Synthesis of N-[5-[4-(3-chloro-2-fluoro-anilino) quinazolin-6-yl]-2-methyl-3-pyridyl] methanesulfonamide (Compound 1)

To a stirred solution of Compound 5a (100 mg, 186.57 µmol, 1 eq) in MeOH (1 mL) was added K₂CO₃ (51.57 mg, 373.14 µmol, 2 eq) at 25° C., and the mixture was stirred at 60° C. for 1 h. LCMS showed starting material was consumed completely and the MS of desired product was detected. The reaction was filtered, then the filtrate was concentrated in vacuum. The crude product was purified by prep-HPLC (column: Phenomenex luna C18 80*40 mm*3 µm; mobile phase: [water(0.04%HCl)-ACN]; B%: 17%-37%,7 min). The desired product (16.75 mg, 33.56 µmol, 17.99% yield, 99.06% purity, HCl) was obtained as a pale yellow solid. ¹H NMR (400 MHz, DMSO-d₆), δ ppm 12.81 (br s, 1 H), 10.09 (br s, 1 H), 9.54 - 9.75 (m, 1 H), 9.21 (s, 1 H), 8.99 (s, 1 H), 8.51 (br d, J=8.76 Hz, 2 H), 8.11 - 8.23 (m, 1 H), 7.66 - 7.73 (m, 1 H), 7.56 - 7.63 (m, 1 H), 7.41 (t, J=8.07 Hz, 1 H), 3.23 (s, 3 H), 2.73 (s, 3 H). MS (M + H) ⁺ = 458.0

### Example 2: Synthesis of N-[5-[4-(3-chloro-2-fluoro-anilino) Quinazolin-6-yl]-2-methoxy-3-pyridyl] Methanesulfonamide (Compound 2)

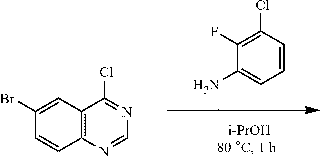

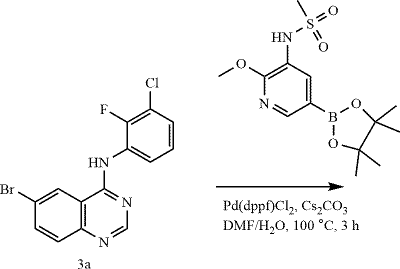

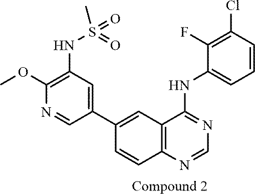

### Step 1: Synthesis of 6-bromo-N-(3-chloro-2-fluoro-phenyl)quinazolin-4-amine (3a)

To a solution of 6-bromo-4-chloro-quinazoline (870 mg, 3.57 mmol, 1 eq) in i-PrOH (30 mL) was added 3-chloro-2-fluoroaniline (520.10 mg, 3.57 mmol, 1 eq), the mixture was stirred at 80° C. for 1 h. LCMS showed starting material was consumed completely and the MS of desired product was detected. The reaction mixture was concentrated in vacuum, and the desired product (1.2 g, 3.40 mmol, 95.25% yield) was obtained as a yellow solid. MS (M + H) ⁺ =353.9

### Step 2: Synthesis of N-[5-[4-(3-chloro-2-fluoro-anilino) quinazolin-6-yl]-2-methoxy-3-pyridyl] methanesulfonamide (Compound 2)

To a solution of Compound 3a (80 mg, 226.89 µmol, 1 eq) in DMF (0.5 mL) and H₂O (0.1 mL) was added Cs₂CO₃ (221.78 mg, 680.68 µmol, 3 eq), Pd(dppf)Cl₂ (16.60 mg, 22.69 µmol, 0.1 eq) and N-[2-methoxy-5-(4, 4, 5, 5-tetramethyl-1, 3, 2-dioxaborolan-2-yl)-3-pyridyl]methanesulfonamide (74.46 mg, 226.89 µmol, 1 eq). The mixture was bubbled with N₂, the reaction was stirred at 100° C. for 3 h. LCMS showed starting material was consumed completely and the MS of desired product was detected. The reaction was filtered, and the filtrate was concentrated in vacuum. The crude product was purified by prep-HPLC (column: Phenomenex Luna 80*30 mm*3 µm; mobile phase: [water (0.04%HCl)-ACN]; B%: 5%-35%, 8 min), and by prep-HPLC(column: Waters Xbridge BEH C18 100*30 mm*10 µm; mobile phase: [water( NH₄HCO₃)-ACN]; B%: 25%-55%,10 min) to obtain the desired product (7.38 mg, 15.57 µmol, 6.86% yield, 100% purity) as a white solid. ¹H NMR (400 MHz, DMSO-d₆) δ ppm 10.13 (s, 1 H), 9.43 (br s, 1 H), 8.78 (s, 1 H), 8.54 (s, 2 H), 8.22 (d, J=8.51 Hz, 1 H), 8.10 (d, J=2.13 Hz, 1 H), 7.90 (d, J=8.63 Hz, 1 H), 7.45 - 7.67 (m, 2 H), 7.29 - 7.40 (m, 1 H), 4.00 (s, 3 H), 3.09 (s, 3 H). MS (M + H)⁺ = 474.1

### Example 3: Synthesis of N-[5-[4-(3-chloro-2-fluoro-anilino) Quinazolin-6-yl]-3-pyridyl] Methanesulfonamide (Compound 3)

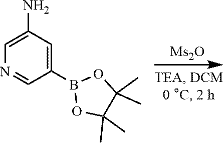

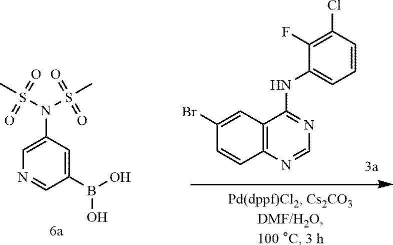

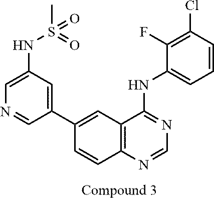

### Step 1: Synthesis of N-methylsulfonyl-N-[5-(4, 4, 5, 5-tetramethyl-1, 3,2-dioxaborolan-2-yl)- 3-pyridyl] methanesulfonamide (6a)

To a solution of 5-(4,4,5,5-tetramethyl-1,3,2-dioxaborolan-2-yl)pyridin-3-amine (200 mg, 908.78 µmol, 1 eq) in DCM (4 mL) was added dropwise TEA (367.83 mg, 3.64 mmol, 505.96 µL, 4 eq) and methylsulfonyl methanesulfonate (633.22 mg, 3.64 mmol, 4 eq). The mixture was purged with N₂, the reaction was stirred at 0° C. for 2 h. LCMS showed starting material was consumed completely and the MS of desired product was detected. The mixture was extracted with ethyl acetate (2 × 30 mL), and the combined organic layers were washed with brine (20 mL), dried over Na₂SO₄, filtered, and concentrated under reduced pressure to give the desired product (300 mg, 797.33 µmol, 87.74% yield) as a brown oil. MS (M + H) ⁺ =295.0

### Step 2: Synthesis of N-[5-[4-(3-chloro-2-fluoro-anilino) quinazolin-6-yl]-3-pyridyl] methanesulfonamide (Compound 3)

To a solution of N-methylsulfonyl-N-[5-(4, 4, 5, 5-tetramethyl-1, 3, 2-dioxaborolan-2-yl)-3-pyridyl] methanesulfonamide (150 mg, 398.66 µmol, 1 eq) in DMF (0.5 mL) and H₂O (0.1 mL) was added Cs₂CO₃ (389.68 mg, 1.20 mmol, 3 eq), Pd(dppf)Cl₂ (29.17 mg, 39.87 µmol, 0.1 eq) and 6-bromo-N-(3-chloro-2-fluoro-phenyl)quinazolin-4-amine (140.56 mg, 398.66 µmol, 1 eq), the mixture was bubbled with N₂, the reaction was stirred at 100° C. for 3 h. LCMS showed starting material was consumed completely and the MS of desired product was detected. The reaction mixture was passed through a filter and the filtrate was concentrated in vacuum. The crude product was then purified by prep-HPLC (column: Phenomenex luna C18 80*40 mm*3 µm; mobile phase: [water (0.04% HCl)-ACN]; B%: 17%-37%, 7 min) to obtain the desired product (19.16 mg, 39.17 µmol, 9.82% yield, 98.19% purity, HCl) as a yellow solid. ¹H NMR (400 MHz, DMSO-d6) δ ppm 12.55 (br s, 1 H), 10.50 (br s, 1 H), 9.38 - 9.54 (m, 1 H), 9.01 (s, 2 H), 8.57 (s, 1 H), 8.51 (br d, J=8.76 Hz, 1 H), 8.11 - 8.23 (m, 2 H), 7.66 - 7.73 (m, 1 H), 7.56 - 7.63 (m, 1 H), 7.41 (t, J=8.07 Hz, 1 H) 3.23 (s, 3 H). MS (M + H)⁺ = 444.0.

### Example 4: Synthesis of N-[2-chloro-5-[4-(3-chloro-2-fluoro-anilino) quinazolin-6-yl]-3-pyridyl] Methanesulfonamide (Compound 6)

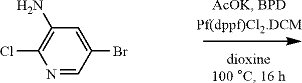

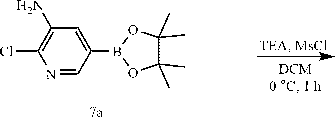

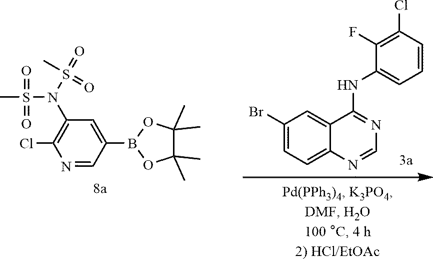

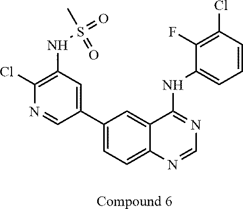

### Step 1: Synthesis of 2-chloro-5-(4, 4, 5, 5-tetramethyl-1, 3, 2-dioxaborolan-2-yl) pyridin-3-amine (7a)

To a stirred solution of 5-bromo-2-chloro-pyridin-3-amine (14 g, 67.48 mmol, 1 eq) in dioxane (200 mL) was added Pd(dppf)Cl₂.CH₂Cl₂ (5.51 g, 6.75 mmol, 0.1 eq), potassium acetate (19.87 g, 202.45 mmol, 3 eq), and BPD (17.14 g, 67.48 mmol, 1 eq). The mixture was purged with N₂ 3 times, and the mixture was stirred at 100° C. for 16 h. The reaction mixture was then cooled to room temperature, quenched with water (80 mL), and extracted with ethyl acetate (2 × 100 mL). The combined organics were washed with brine (80 mL), dried over Na₂SO₄, filtered and concentrated under reduced pressure, and the residue was purified by MPLC (ISCO 80 g silica, 10-60 % ethyl acetate in petroleum ether, gradient over 30 min). The desired product (4 g, 15.72 mmol, 23.29% yield) was obtained as a yellow solid. ¹H NMR (400 MHz, DMSO-d6) δ ppm 7.77 (s, 1 H), 7.41 (s, 1 H), 5.56 (s, 2 H), 1.28 (s, 12 H). MS (M + H)⁺ = 255.2

### Step 2: Synthesis of N-[2-chloro-5-(4, 4, 5, 5-tetramethyl-1, 3, 2-dioxaborolan-2-yl) -3-pyridyl] -N-methylsulfonyl-methanesulfonamide (8a)

To a solution of 2-chloro-5-(4, 4, 5, 5-tetramethyl-1, 3, 2-dioxaborolan-2-yl)pyridin-3-amine (4 g, 15.72 mmol, 1 eq) in DCM (35 mL) was added dropwise TEA (6.36 g, 62.86 mmol, 8.75 mL, 4 eq) and MsCl (6.790 g, 59.27 mmol, 4.59 mL, 3.77 eq), the mixture was purged with N₂, and then stirred at 0° C. for 1 h. TLC (Petroleum ether/Ethyl acetate=3:1, R_{f=}0.84) showed starting material was consumed completely and new spot was formed. The reaction mixture was concentrated in vacuum, and the residue was poured into MeOH (10 mL). The mixture was then stirred at 20° C. for 1 h, passed through a filter, and the filter cake was dried in vacuum to give the desired compound (4.2 g, 10.23 mmol, 65.07% yield) as a pale green solid. ¹H NMR (400 MHz, DMSO-d₆) δ ppm 8.65 (d, J=1.71 Hz, 1 H), 8.26 (d, J=1.71 Hz, 1 H), 3.63 (s, 6 H), 1.33 (s, 12 H).

### Step 3: Synthesis of N-[2-chloro-5-[4-(3-chloro-2-fluoro-anilino) quinazolin-6-yl]-3-pyridyl] methanesulfonamide (Compound 6)

To a stirred solution of Compound 3a (5.41 g, 15.34 mmol, 1 eq) in DMF (8 mL) and H₂O (1.3 mL) was added K₃PO₄ (9.77 g, 46.02 mmol, 3 eq), Pd(PPh₃)₄ (1.77 g, 1.53 mmol, 0.1 eq) and N-[2-chloro-5-(4, 4, 5, 5-tetramethyl-1, 3, 2-dioxaborolan-2-yl)-3-pyridyl]-N-methylsulfonyl-methanesulfonamide (6.3 g, 15.34 mmol, 1 eq) the mixture was purged with N₂ three times, and was then stirred at 100° C. for 4 h under N₂. LCMS showed starting material was consumed completely and the MS of desired product was detected. The reaction mixture was passed through a filter, and the filtrate was concentrated in vacuum. The crude product was purified by prep-HPLC (column: Xtimate C18 10 u 250 mm*80 mm; mobile phase: [water (10 mm NH₄HCO₃)-ACN]; B%: 20%-50%, 22 min) to afford the desired product as free base. The free base was dissolved in Ethyl acetate (30 mL), then 4 M HCl in Ethyl acetate was added and the mixture was stirred at 25° C. for 1 h. The reaction mixture was then concentrated in vacuum to afford Compound 6-HCl (4.79 g, 8.91 mmol, 58.06% yield, 95.71% purity, HCl) as a yellow solid. ¹H NMR (400 MHz, DMSO-d₆) δ ppm 12.30 (br s, 1 H), 9.99 (br s, 1 H), 9.26 - 9.54 (m, 1 H), 8.93 - 8.99 (m, 1 H), 8.89 (dd, J=4.71, 2.38 Hz, 1 H), 8.49 - 8.57 (m, 1 H), 8.35 - 8.41 (m, 1 H), 8.08 (br t, J=9.35 Hz, 1 H), 7.64 - 7.70 (m, 1 H), 7.54 - 7.62 (m, 1 H), 7.32 - 7.43 (m, 1 H), 3.16 - 3.28 (m, 3 H). MS (M + H)⁺ = 477.9

### Example 5: Synthesis of N-[2-chloro-5-[4-(3-chloro-2-fluoro-anilino)-3-cyano-6-quinolyl]-3-pyridyl]methanesulfonamide (Compound 10)

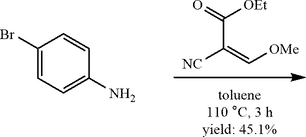

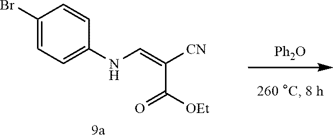

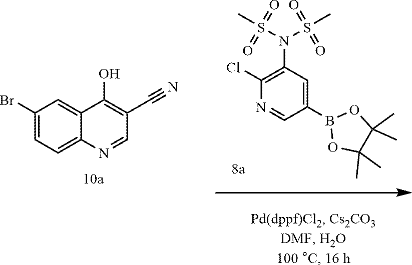

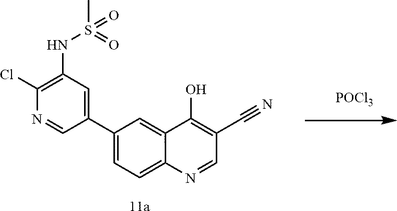

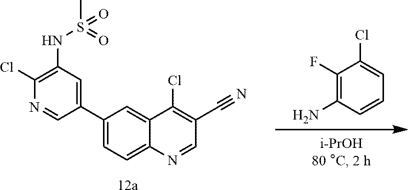

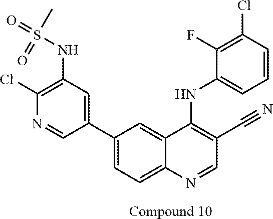

### Step 1: Synthesis of ethyl (Z)-3-(4-bromoanilino)-2-cyano-prop-2-enoate (9a)

A solution of 4-bromoaniline (184.88 g, 1.07 mol, 1 eq) in toluene (1.5 L) was added ethyl (E)-2-cyano-3-ethoxy-prop-2-enoate (200 g, 1.18 mol, 1.1 eq), and the mixture was stirred at 110° C. for 6 h. TLC (Petroleum ether/Ethyl acetate=3:1, R_{f}=0.88) showed starting material was consumed completely and new spot was formed. The reaction mixture was passed through a filter, and filter cake was dried in vacuum to give the desired product (143 g, 484.53 mmol, 45.08% yield) as a white solid. ¹H NMR (400 MHz, CHLOROFORM-d) δ = 10.74 (br d, J=13.0 Hz, 1 H), 8.68 - 7.78 (m, 1 H), 7.54 - 7.46 (m, 2 H), 7.10 - 6.96 (m, 2 H), 4.35 - 4.21 (m, 2 H), 1.35 (td, J=7.1, 9.5 Hz, 3 H).

### Step 2: Synthesis of 6-bromo-4-hydroxy-quinoline-3-carbonitrile (10a)

A solution of Compound 9a (23 g, 77.93 mmol, 1 eq) in Ph₂0 (200 mL) was stirred at 270° C. for 8 h. TLC (Petroleum ether/Ethyl acetate=3:1, R_{f}=0.43) showed a minor amount of starting material remained and a new spot was formed. The reaction mixture was poured into MTBE (200 mL), passed through a filter, and filter cake was dried in vacuum to give the desired product (38.16 g, crude) as a brown solid. ¹H NMR (400 MHz, DMSO-d₆) δ = 12.96 (br s, 1 H), 8.76 (s, 1 H), 8.16 (br s, 1 H), 7.99 - 7.85 (m, 1 H), 7.58 (br d, J=8.8 Hz, 1 H).

### Step 3: Synthesis of N-[2-chloro-5-(3-cyano-4-hydroxy-6-quinolyl)-3-pyridyl]methanesulfonamide (11a)

To a stirred solution of Compound 8a (4 g, 9.74 mmol, 1 eq) in dioxane (30 mL) and H₂O (6 mL) was added Compound 10a (3.15 g, 12.66 mmol, 1.3 eq), Cs₂CO₃ (9.52 g, 29.22 mmol, 3 eq), and Pd(dppf)Cl₂ (712.64 mg, 973.94 µmol, 0.1 eq). Nitrogen gas was bubbled through the mixture, and then the mixture was stirred at 100° C. for 2 h. LCMS showed the starting material was consumed completely, and the desired MS was detected. The reaction mixture was then passed through a filter, and the filtrate was concentrated and purified by prep-HPLC (column: Agela DuraShell C18 250*70 mm*10 µm; mobile phase: [water (10 mM NH₄HCO₃)-ACN]; B%: 1%-20%,20 min) to give the desired product (2.52 g, 6.72 mmol, 69.03% yield) as a yellow solid. MS (M + H)⁺ =375.0

### Step 4: Synthesis of N-[2-chloro-5-(4-chloro-3-cyano-6-quinolyl)-3-pyridyl]methanesulfonamide (12a)

To a solution of POCl₃ (18 mL) was added N-[2-chloro-5-(3-cyano-4-hydroxy-6-quinolyl)-3-pyridyl]methanesulfonamide (2.5 g, 6.67 mmol, 1 eq), the mixture was purged with nitrogen gas, and stirred at 130° C. for 16 h. LCMS showed the starting material was consumed completely, and desired MS was detected. The reaction mixture was concentrate in vacuum, and the residue was then re-dissolved in ethyl acetate (10 mL), and poured into water (20 mL). The aqueous phase was extracted with ethyl acetate (2 × 20 mL), and the combined organic phases were dried with anhydrous Na₂SO₄, filtered and concentrated in vacuum. The crude product was purified by flash column (ISCO 20 g silica, 70% ethyl acetate in petroleum ether, gradient over 20 min) to provide the desired product (1.2 g, 3.05 mmol, 45.75% yield) as a yellow solid. ¹H NMR (400 MHz, DMSO-d₆) δ = 9.95 (s, 1 H), 9.26 (s, 1 H), 8.82 (d, J=2.3 Hz, 1 H), 8.57 (d, J=1.9 Hz, 1 H), 8.45 - 8.42 (m, 1 H), 8.36 - 8.32 (m, 1 H), 8.31 (d, J=2.3 Hz, 1 H), 3.21 (s, 3 H). MS (M + H)⁺ =393.0

### Step 5: Synthesis of N-[2-chloro-5-[4-(3-chloro-2-fluoro-anilino)-3-cyano-6-quinolyl]-3-pyridyl]methanesulfonamide (Compound 10)

To a solution of Compound 12a (70 mg, 178.01 µmol, 1 eq) in i-PrOH (1 mL) was added 3-chloro-2-fluoro-aniline (25.91 mg, 178.01 µmol, 1 eq). The mixture was stirred at 80° C. for 2 h. LCMS showed the starting material was consumed completely, and desired MS was detected. The reaction mixture was concentrated in vacuum, and the crude product was purified by prep-HPLC (column: Phenomenex luna C18 80*40 mm*3 µm; mobile phase: [water (0.04%HCl)-ACN]; B%: 31%-49%,7 min) to give the desired product (27.77 mg, 51.54 µmol, 28.95% yield, 100% purity, HCl) as a yellow solid. ¹H NMR (400 MHz, DMSO-d6) δ = 9.97 (s, 1H), 9.09 (br s, 1 H), 8.90 (s, 1 H), 8.86 (d, J=2.0 Hz, 1 H), 8.41 - 8.31 (m, 2 H), 8.12 (br d, J=8.8 Hz, 1 H), 7.64 (br t, J=6.9 Hz, 1 H), 7.55 (br t, J=6.8 Hz, 1 H), 7.40 - 7.32 (m, 1 H), 3.20 (s, 3 H). MS (M + H)⁺ = 501.9.

### Example 6: The Z′-LYTE® Biochemical Assay

The Z′-LYTE® biochemical assay employs a fluorescence-based, coupled-enzyme format and is based on the differential sensitivity of phosphorylated and non-phosphorylated peptides to proteolytic cleavage. The peptide substrate is labeled with two fluorophores—one at each end—that make up a FRET pair. In the primary reaction, the kinase transfers the gamma-phosphate of ATP to a single tyrosine, serine or threonine residue in a synthetic FRET-peptide. In the secondary reaction, a site-specific protease recognizes and cleaves non-phosphorylated FRET-peptides. Phosphorylation of FRET-peptides suppresses cleavage by the Development Reagent. Cleavage disrupts FRET between the donor (i.e., coumarin) and acceptor (i.e., fluorescein) fluorophores on the FRET-peptide, whereas uncleaved, phosphorylated FRET-peptides maintain FRET. A ratiometric method, which calculates the ratio (the Emission Ratio) of donor emission to acceptor emission after excitation of the donor fluorophore at 400 nm, is used to quantitate reaction progress.

A significant benefit of this ratiometric method for quantitating reaction progress is the elimination of well-to-well variations in FRET-peptide concentration and signal intensities. As a result, the assay yields very high Z′-factor values (>0.7) at a low percent phosphorylation.

Both cleaved and uncleaved FRET-peptides contribute to the fluorescence signals and therefore to the Emission Ratio. The extent of phosphorylation of the FRET-peptide can be calculated from the Emission Ratio. The Emission Ratio will remain low if the FRET-peptide is phosphorylated (i.e., no kinase inhibition) and will be high if the FRET-peptide is non-phosphorylated (i.e., kinase inhibition).

Enzyme: The ADAPTA universal kinase assay is a homogenous, fluorescent based immunoassay for the detection of ADP. In contrast to ATP depletion assays, the ADAPTA assay is extremely sensitive to ADP formation such that a majority of the signal change occurs in the first 10-20% conversion of ATP to ADP. This makes the ADAPTA universal kinase assay ideally suited for use with low activity kinases.

The principle of the ADAPTA universal kinase assay is outlined below. The assay itself can be divided into two phases: a kinase reaction phase, and an ADP detection phase. In the kinase reaction phase, all components required for the kinase reaction are added to the well, and the reaction is allowed to incubate for 60 minutes. After the reaction, a detection solution consisting of a europium labeled anti-ADP antibody, an Alexa Fluor® 647 labeled ADP tracer, and EDTA (to stop the kinase reaction) is added to the assay well. ADP formed by the kinase reaction (in the absence of an inhibitor) will displace the Alexa Fluor® 647 labeled ADP tracer from the antibody, resulting in a decrease in the TR-FRET signal. In the presence of an inhibitor, the amount of ADP formed by the kinase reaction is reduced, and the resulting intact antibody-tracer interaction results in a high TR-FRET signal.

### Z′-LYTE® Assay Conditions

Test Compounds: The Test Compounds are screened in 1% DMSO (final) in the well. For 10 point titrations, 3-fold serial dilutions are conducted from the starting concentration of the customer’s choosing.

Peptide/Kinase Mixtures: All Peptide/Kinase Mixtures are diluted to a 2X working concentration in the appropriate Kinase Buffer.

ATP Solution: All ATP Solutions are diluted to a 4X working concentration in Kinase Buffer (50 mM HEPES pH 7.5, 0.01% BRIJ-35, 10 mM MgCl₂,1 mM EGTA). ATP Km apparent is previously determined using a Z′-LYTE® assay.

Development Reagent Solution: The Development Reagent is diluted in Development Buffer.

10X Novel PKC Lipid Mix: 2 mg/mL Phosphatidyl Serine, 0.2 mg/mL DAG in 20 mM HEPES, pH 7.4, 0.3% CHAPS. For 5 mL 10X Novel PKC Lipid Mix: 1. Add 10 mgs Phosphatidyl Serine (Avanti Polar Lipids Part# 8400032C or 840039C) and 1 mg DAG (Avanti Polar Lipids Part# 800811C) to a glass tube. 2. Remove the chloroform from lipid mixture by evaporating to a clear, thin film under a stream of nitrogen. Continuous rotation of the tube, at an angle to ensure maximum surface area of the lipid solution, will promote the thinnest film. 3. Add 5 mL resuspension buffer, 20 mM HEPES, 0.3% CHAPS, pH 7.4, to the dried lipid mix 4. Heat gently to 50-60° C. for 1-2 minutes and vortex in short intervals until the lipids are dissolved to a clear or slightly hazy solution. The lipids are typically in solution after 2-3 heat/vortex cycles. 5. Cool to room temperature, aliquot into single use volumes and store at -20° C.

Assay Protocol: Bar-coded Corning, low volume NBS, black 384-wellplate (Corning Cat. #4514) 1. 2.5 µL - 4X Test Compound or 100 nL 100X plus 2.4 µL kinase buffer. 2. 5 µL - 2X Peptide/Kinase Mixture. 3. 2.5 µL - 4X ATP Solution. 4. 30-second plate shake. 5. 60-minute Kinase Reaction incubation at room temperature. 6. 5 µL - Development Reagent Solution. 7. 30-second plate shake. 8. 60-minute Development Reaction incubation at room temperature. 9. Read on fluorescence plate reader and analyze the data.

ADP formation is determined by calculating the emission ratio from the assay well. The emission ratio is calculated by dividing the intensity of the tracer (acceptor) emission by the intensity of the Eu (donor) emission at 615 nm as shown in the equation below.

Since the ADAPTA technology measures ADP formation (i.e. conversion of ATP to ADP) it can be used to measure any type of ATP hydrolysis, including intrinsic ATPase activity of kinases. In this case, the substrate is water, not a lipid or peptide. The SelectScreen® service screens CHUK in this way, so a substrate is not included in the kinase reaction. A reference for using intrinsic ATPase activity to screen for kinase inhibitors is provided below.

### Adapta® Assay Conditions

Test Compounds: The Test Compounds are screened in 1% DMSO (final) in the well. For 10 point titrations, 3-fold serial dilutions are conducted from the starting concentration of the customer’s choosing.

Substrate/Kinase Mixtures: All Substrate/Kinase Mixtures are diluted to a 2X working concentration in the appropriate Kinase Buffer (see section Kinase Specific Assay Conditions for a complete description).

ATP Solution: All ATP Solutions are diluted to a 4X working concentration in water. ATP Km apparent is previously determined using a radiometric assay except when no substrate is available in which case an Adapta® assay is conducted.

Detection Mix: The Detection Mix is prepared in TR-FRET Dilution Buffer. The Detection mix consists of EDTA (30 mM), Eu-anti-ADP antibody (6 nM) and ADP tracer. The detection mix contains the EC60 concentration of tracer for 5-150 µM ATP.

Assay Protocol: Bar-coded Corning, low volume, white 384-well plate (Corning Cat. #4512)1. 2.5 µL - 4X Test Compound in 30 mM HEPES or 100 nL 100X in 100% DMSO plus 2.4 µL 30 mM HEPES. 2. 2.5 µL - 4X ATP Solution. 3. 5 µL - 2X Substrate/Kinase Mixture. 4. 30-second plate shake. 5. 1-minute centrifuge at 1000 × g. 6. 60-minute Kinase Reaction incubation at room temperature. 7. 5 µL - Detection Mix. 8. 30-second plate shake. 9. 1-minute centrifuge at 1000 × g. 10. 60-minute Detection Mix equilibration at room temperature. 11. Read on fluorescence plate reader and analyze the data.

The affinity for PI3Ka and EGFR enzymes of selected compounds of the invention, presented as the 50% inhibitory concentration (IC₅₀) and percent inhibition at 100 nM, are provided in Table 3 below with Comparative Compound 1. The IC₅₀ data in Table 2 is presented as “++++” (value is 2 nM or less), “+++” (value is greater than 2 nM and less than or equal to 20 nM), “++” (value is greater than 20 nM and less than or equal to 200 nM) and “+” (value is greater than 200 nM). The % inhibition at 100 nM data presented in Table 3 is presented as “*” (value is 10% or less), “**” (value is greater than 10% and less than or equal to 80%), “***” (value is greater than 80% and less than or equal to 90%) and “****” (value is greater than 90%). Blank is not tested.

TABLE 2

Potency of inhibitory activity of Compound 6 on HER family enzymes

| | |
|---|---|
| HER Family Enzyme | IC₅₀ (nM) |
| EGFR | ++++ |
| HER2 | + |
| HER4 | + |
| PI3Kα | ++++ |
| PI3Kβ | ++ |
| P13Kγ | ++++ |
| P13Kδ | ++++ |
| DNA-PK | ++++ |
| MTOR | +++ |

TABLE 3

Percent Inhibition of target enzymes for molecules at a concentration of 100 nM/liter

| | | | |
|---|---|---|---|
| | PI3K A | EGFR | DNA-PK |
| Compound 1 | *** | **** | **** |
| Compound 2 | **** | **** | *** |
| Compound 3 | ** | **** | *** |
| Compound 6 | **** | **** | **** |
| Compound 10 | **** | **** | **** |

### Example 7: Assessment of Metabolic Stability in Liver Microsomes

Working solution: 5 µL of compound and control stock solution (10 mM in dimethyl sulfoxide (DMSO)) were diluted with 495 µL of acetonitrile (ACN) (intermediate solution concentration: 100 µM, 99% ACN)

NADPH Cofactor Preparation: NADPH powder: β-Nicotinamide adenine dinucleotide phosphate reduced form, tetrasodium salt; NADPH 4 Na. The appropriate amount of NADPH powder was weighed and diluted into a 10 mM MgCl2 solution (working solution concentration: 10 unit/mL; final concentration in reaction system: 1 unit/mL)

Liver Microsomes Preparation: The appropriate concentrations of microsome working solutions were prepared in 100 mM potassium phosphate buffer. Cold (4° C.) acetonitrile (ACN) containing 200 ng/mL tolbutamide and 200 ng/mL labetalol as internal standards (IS) was used as the stop solution

Assay Procedure: Pre-warm empty ‘Incubation’ plates T60 and NCF60 for 10 minutes. Dilute liver microsomes to 0.56 mg/mL in 100 mM phosphate buffer. Transfer 445 µL microsome working solutions (0.56 mg/mL) into pre-warmed ‘Incubation’ plates T60 and NCF60, Then pre-incubate ‘Incubation’ plates T60 and NCF60 for 10 min at 37° C. with constant shaking. Transfer 54 µL liver microsomes to blank plate, then add 6 µL NAPDH cofactor to blank plate, and then add 180 µL quenching solution to blank plate. Add 5 µL compound working solution (100 µM) into ‘incubation’ plates (T60 and NCF60) containing microsomes and mix 3 times thoroughly.

For the NCF60 plate, add 50 µL of buffer and mix 3 times thoroughly. Start timing; plate will be incubated at 37° C. for 60 min while shaking. In ‘Quenching’ plate T0, add 180 µL quenching solution and 6 µL NAPDH cofactor. Ensure the plate is chilled to prevent evaporation. For the T60 plate, mix 3 times thoroughly, and immediately remove 54 µL mixture for the 0-min time point to ‘Quenching’ plate. Then add 44 µL NAPDH cofactor to incubation plate (T60). Start timing; plate will be incubated at 37° C. for 60 min while shaking. At 5, 10, 20, 30, and 60 min, add 180 µL quenching solution to ‘Quenching’ plates, mix once, and serially transfer 60 µL sample from T60 plate per time point to ‘Quenching’ plates. For NCF60: mix once, and transfer 60 µL sample from the NCF60 incubation to ‘Quenching’ plate containing quenching solution at the 60-min time point. All sampling plates are shaken for 10 min, then centrifuged at 4000 rpm for 20 minutes at 4° C. Transfer 80 µL supernatant into 240 µL HPLC water, and mix by plate shaker for 10 min. Each bioanalysis plate was sealed and shaken for 10 minutes prior to LC-MS/MS analysis.

The equation of first order kinetics was used to calculate T1/2 and CLint(mic) (µL/min/mg). Equation of first order kinetics:

$${\text{C}}_{\text{t}} {\text{=C}}_{\text{0}} \cdot {\text{e}}^{- {\text{k}}_{\text{e}} \cdot \text{t}}$$

$$\text{when} {\text{C}}_{\text{t}} \text{=} \frac{\text{1}}{\text{2}} {\text{C}}_{\text{0}} ,$$

$${\text{T}}_{1 / 2} = \frac{\text{Ln2}}{{\text{k}}_{\text{e}}} = \frac{0.693}{{\text{k}}_{\text{e}}}$$

$$\begin{matrix}{\text{CL}}_{\text{int} \left(\text{mic}\right)} = \frac{0.693}{\text{In} \text{vitro} {\text{T}}_{1 / 2}} \cdot \\ \frac{\text{1}}{\text{mg} / \text{mL} \text{microsomal} \text{protein} \text{in} \text{reaction} \text{system}}\end{matrix}$$

$${\text{CL}}_{\text{int} \left(\text{liver}\right)} = {\text{CL}}_{{\text{int}}_{\left(\text{mic}\right)}} \cdot \frac{\text{mg} \text{microsomes}}{\text{g} \text{liver}} \cdot \frac{\text{g} \text{liver}}{\text{kg} \text{body} \text{weight}}$$

The biological stability of the compounds of the invention can be measured by determining its ½ life in the presence microsomes. Presented in Table 4, is the half-life of selected compounds of the invention in the presence of human liver microsomes (HLM) or mouse liver microsomes (MLM) as described above. In Table 4, half-life is presented as “++++” (value is greater than 200 minutes), “+++” (value is greater than 100 minutes and less than or equal to 200 minutes), “++” (value is greater than 10 minutes and less than or equal to 100 minutes) and “+” (value is 10 minutes or less). Blank is not tested.

TABLE 4

Microsome stability of Compound 6

| | |
|---|---|
| Microsome | Measure |
| Mouse Liver Microsome (MLM) | ++ |
| Human Liver Microsome (HLM) | ++ |

### Example 8: Solubility Assessment

Preparation of stock solutions: The stock solutions of test compounds and control compound diclofenac were prepared in DMSO at the concentrations of 10 mM.

Procedure for solubility determination: 15 µL of stock solution (10 mM) of each sample was placed in order into their proper 96-well rack. 485 µL of (SIF, SGF, PBS 7.4, FESSIF, or FESSGF) was added into each vial of the cap-less Solubility Sample plate. The assay was performed in duplicate. Add one stir stick to each vial and seal using a molded PTFE/Silicone plug. Then the solubility sample plate was transferred to the Eppendorf Thermomixer Comfort plate shaker and shaken at 25° C. at 1100 rpm for 2 hours. After completion of the 2 hours, plugs were removed and the stir sticks were removed using a big magnet, the samples from the Solubility Sample plate were transferred into the filter plate. Using the Vacuum Manifold, all the samples were filtered. Aliquot of 5 µL and 5 µL DMSO were taken from the filtrate followed by addition of 490 µL of a mixture of H₂O and acetonitrile containing internal standard (1:1). A certain proportion of ultrapure water was used to dilute the diluent according to the peak shape. The dilution factor was changed according to the solubility values and the LC-MS signal response.

Preparation of 300 µM standards (STD): From the 10 mM DMSO STD plate, 6 µL was transferred into the remaining empty plate, and then 194 µL of DMSO was added to that plate to have a STD concentration of 300 µM. From the 300 µM DMSO STD plate, 5 µL DMSO STD and 5 µL SIF was transferred into the remaining empty plate, and then 490 µL of a mixture of H2O and acetonitrile containing internal standard (1:1) was added to that plate to have a final STD concentration of 3 µM. A certain proportion of ultrapure water was used to dilute the diluent according to the peak shape. The concentrations of the standard samples were changed according to the LC-MS signal response.

Procedure for sample analysis: The plate was placed into the well plate autosampler. The samples were evaluated by LC-MS/MS analysis.

Data analysis: All calculations were carried out using Microsoft Excel. The filtrate was analyzed and quantified against a standard of known concentration using LC coupled with mass spectral peak identification and quantitation. Solubility values of the test compound and control compound were calculated as follows:

$$\left[\text{Sample}\right] = \frac{A r e a r a t i {o}_{S a m p l e} \times I N J V O L S T D \times D {F}_{S a m p l e} \times \left[S T D\right]}{A r e a r a t i o S T D \times I N J V O {L}_{S a m p l e}}$$

Any value of the compounds that was not within the specified limits was rejected and the experiment was repeated.

The solubility data for selected compounds of the invention is provided in Table 5. The solubility data in Table 5 is presented as “++++” (value is greater than 200 µM), “+++” (value is greater than 100 µM and less than or equal to 200 µM), “++” (value is greater than 5 µM and less than or equal to 100 µM) and “+” (value is 5 µM or less).

TABLE 5

Solubility data for Compound 6 in various aqueous medium.

| | |
|---|---|
| Solvent | Solubility (µM) |
| pH 7.4 Buffer | ++ |
| Simulated Intestinal Fluid (SIF) | ++ |
| Simulated Gastric Fluid (SGF) | ++++ |

### Example 9: NCI COMPARE Panel

The human tumor cell lines of the cancer screening panel are grown in RPMI 1640 medium containing 5% fetal bovine serum and 2 mM L-glutamine. For a typical screening experiment, cells are inoculated into 96 well microtiter plates in 100 µL at plating densities ranging from 5,000 to 40,000 cells/well depending on the doubling time of individual cell lines. After cell inoculation, the microtiter plates are incubated at 37° C., 5% CO2, 95% air and 100% relative humidity for 24 h prior to addition of experimental drugs.

After 24 h, two plates of each cell line are fixed in situ with TCA, to represent a measurement of the cell population for each cell line at the time of drug addition (Tz). Experimental drugs are solubilized in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 µg/ml gentamicin. Additional four, 10-fold or ½ log serial dilutions are made to provide a total of five drug concentrations plus control. Aliquots of 100 µL of these different drug dilutions are added to the appropriate microtiter wells already containing 100 µL of medium, resulting in the required final drug concentrations.

Following drug addition, the plates are incubated for an additional 48 h at 37° C., 5 % CO₂, 95% air, and 100% relative humidity. For adherent cells, the assay is terminated by the addition of cold TCA. Cells are fixed in situ by the gentle addition of 50 µL of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 µL) at 0.4 % (w/v) in 1% acetic acid is added to each well, and plates are incubated for 10 minutes at room temperature. After staining, unbound dye is removed by washing five times with 1% acetic acid and the plates are air dried. Bound stain is subsequently solubilized with 10 mM trizma base, and the absorbance is read on an automated plate reader at a wavelength of 515 nm. For suspension cells, the methodology is the same except that the assay is terminated by fixing settled cells at the bottom of the wells by gently adding 50 µL of 80% TCA (final concentration, 16% TCA). Using the seven absorbance measurements [time zero, (Tz), control growth, (C), and test growth in the presence of drug at the five concentration levels (Ti)], the percentage growth is calculated at each of the drug concentrations levels. Percentage growth inhibition is calculated as:

$$\left[\left(\text{Ti-Tz}\right) / \left(\text{C-Tz}\right)\right] \text{x 100 for concentrations for which} \text{Ti>} / \text{=Tz}$$

$$\left[\left(\text{Ti-Tz}\right) / \text{Tz}\right] \text{x 100 for concentrations for which Ti} < \text{Tz}$$

Growth inhibition of 50% (GI₅₀) is calculated from [(Ti-Tz)/(C-Tz)] ×× 100 = 50, which is the drug concentration resulting in a 50% reduction in the net protein increase (as measured by SRB staining) in control cells during the drug incubation.

The efficacy of selected compounds of the invention to inhibit tumor growth, reported as GI₅₀ in various cell lines, is provided in Table 6 below. The GI₅₀ data in Table 6 is presented as “****” (value is 75 nM or less), “***” (value is greater than 75 nM and less than or equal to 125 nM), “**” (value is greater than 125 nM and less than or equal to 250 nM) and “*” (value is greater than 250 nM). Table 6: NCI-60 Cancer Cell Panels with GI₅₀ for Compounds 6 & 10

| | | | |
|---|---|---|---|
| Panel | Cell Line | Compound 6 GI₅₀ (nM) | Compound 10 GI₅₀ (nM) |
| Breast Cancer | MCF7 | **** | **** |
| Breast Cancer | MDA-MB-231/ATCC | ** | * |
| Breast Cancer | HS 578T | **** | **** |
| Breast Cancer | BT-549 | *** | **** |
| Breast Cancer | T-47D | **** | ** |
| Breast Cancer | MDA-MB-468 | ** | **** |
| CNS Cancer | SF-268 | ** | *** |
| CNS Cancer | SF-295 | ** | ** |
| CNS Cancer | SF-539 | ** | ** |
| CNS Cancer | SNB-19 | ** | ** |
| CNS Cancer | SNB-75 | **** | **** |
| CNS Cancer | U251 | * | **** |
| Colon Cancer | COLO 205 | ** | ** |
| Colon Cancer | HCC-2998 | ** | * |
| Colon Cancer | HCT-116 | ** | **** |
| Colon Cancer | HCT-15 | ** | *** |
| Colon Cancer | HT29 | ** | * |
| Colon Cancer | KM12 | * | * |
| Colon Cancer | SW-620 | * | **** |
| Leukemia | CCRF-CEM | ** | **** |
| Leukemia | HL-60(TB) | *** | **** |

| | | | |
|---|---|---|---|
| Panel | Cell Line | Compound 6 GI₅₀ (nM) | Compound 10 GI₅₀ (nM) |
| Leukemia | K-562 | * | *** |
| Leukemia | MOLT-4 | ** | *** |
| Leukemia | RPMI-8226 | ** | **** |
| Leukemia | SR | * | **** |
| Melanoma | LOX IMVI | ** | *** |
| Melanoma | MALME-3M | **** | *** |
| Melanoma | M14 | ** | * |
| Melanoma | MDA-MB-435 | ** | ** |
| Melanoma | SK-MEL-2 | ** | **** |
| Melanoma | SK-MEL-28 | *** | ** |
| Melanoma | SK-MEL-5 | ** | **** |
| Melanoma | UACC-257 | ** | * |
| Melanoma | UACC-62 | **** | *** |
| Non-Small Cell Lung Cancer | A549/ATCC | ** | * |
| Non-Small Cell Lung Cancer | EKVX | *** | * |
| Non-Small Cell Lung Cancer | HOP-62 | *** | ** |
| Non-Small Cell Lung Cancer | HOP-92 | **** | **** |
| Non-Small Cell Lung Cancer | NCI-H226 | * | * |
| Non-Small Cell Lung Cancer | NCI-H23 | ** | ** |
| Non-Small Cell Lung Cancer | NCI-H322M | ** | * |

| | | | |
|---|---|---|---|
| Panel | Cell Line | Compound 6 GI₅₀ (nM) | Compound 10 GI₅₀ (nM) |
| Non-Small Cell Lung Cancer | NCI-H460 | * | * |
| Non-Small Cell Lung Cancer | NCI-H522 | ** | * |
| Ovarian Cancer | IGROV1 | **** | *** |
| Ovarian Cancer | OVCAR-3 | *** | * |
| Ovarian Cancer | OVCAR-4 | * | * |
| Ovarian Cancer | OVCAR-5 | ** | * |
| Ovarian Cancer | OVCAR-8 | * | * |
| Ovarian Cancer | NCI/ADR-RES | * | * |
| Ovarian Cancer | SK-OV3 | *** | * |
| Prostate Cancer | PC-3 | ** | ** |
| Prostate Cancer | DU-145 | ** | ** |
| Renal Cancer | 786-0 | ** | * |
| Renal Cancer | A498 | **** | **** |
| Renal Cancer | ACHN | ** | * |
| Renal Cancer | CAKI-1 | **** | **** |
| Renal Cancer | RXF 393 | *** | **** |
| Renal Cancer | SN12C | ** | *** |
| Renal Cancer | TK-10 | **** | *** |
| Renal Cancer | UO-31 | **** | ** |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A compound of Formula I: 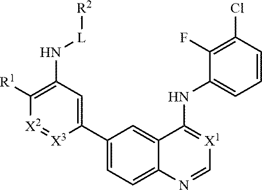
or a pharmaceutically acceptable salt thereof, wherein
X¹ is selected from N or CR³, wherein R³ can be hydrogen, CN, NO₂, NH₂, OH, COOH, halo, C₁₋₆ alkyl, O—C₁₋₆ alkyl, C(O)C₁₋₆ alkyl, and C(O)OC₁₋₆ alkyl, wherein each C₁₋₆ alkyl is optionally substituted;
X² and X³ are each independently selected from N and CH;
L is a bond or a bivalent linking moiety selected from C₁₋₆ alkylene, —C(O)—, —C(O)O—, —S—, —O—, —NR⁴—, —S(O)₂—, —NHS(O)₂—, and —C(O)NH—;
R¹ is selected from hydrogen, C₁₋₆ alkyl, C₃₋₆ cycloalkyl, a 3-7 membered heterocycloalkyl, phenyl, a 5-6 membered heteroaryl, CN, NO₂, NH₂, OH, COOH, halo, O—C₁₋₆ alkyl, C(O)C₁₋₆ alkyl, and C(O)OC₁₋₆ alkyl, wherein each C₁₋₆ alkyl, C₃₋₆ cycloalkyl, 3-7 membered heterocycloalkyl, phenyl, and 5-6 membered heteroaryl is optionally and independently substituted; and
R² is selected from hydrogen, a C₁₋₆ alkyl, a C₃₋₆ cycloalkyl, a 3-7 membered heterocycloalkyl, phenyl, and a 5-6 membered heteroaryl, each of which is optionally and independently substituted.

2. The compound or salt thereof according to claim 1, wherein R³can be hydrogen, CN, NH₂, OH, COOH, halo, C₁₋₆ alkyl, O—C₁₋₆ alkyl, C(O)C₁₋₆ alkyl, and C(O)OC₁₋₆ alkyl, wherein each C₁₋₆ alkyl is optionally substituted.

3. (canceled)

4. (canceled)

5. The compound or salt thereof according to claim 1, wherein X¹ is CR³.

6. The compound or salt thereof according to claim 1, wherein X¹ is N.

7. The compound or salt thereof according to claim 1, wherein at least one of X² and X³ is N.

8. (canceled)

9. The compound or salt thereof according to claim 1, wherein X² is N, and X³ is CH.

10. The compound or salt thereof according to claim 1, wherein both of X² and X³ are CH.

11. (canceled)

12. (canceled)

13. (canceled)

14. (canceled)

15. The compound or salt thereof according to claim 1, wherein R¹ is selected from hydrogen, methyl, ethyl, propyl, methoxy, trifluoromethyl, and halo.

16. (canceled)

17. (canceled)

18. (canceled)

19. (canceled)

20. (canceled)

21. The compound or salt thereof according to claim 1, wherein L is —S(O)₂—.

22. (canceled)

23. The compound or salt thereof according to claim 1, wherein R² is selected from hydrogen, a C₁₋₆ alkyl, optionally and independently substituted with one or more of halo, OH, —NH₂, —N(H)CH₃, —N(CH₃)₂, a C₁₋₆ alkyl, a C₃₋₆ cycloalkyl, a 3-7 membered heterocycloalkyl, phenyl, or a 5-6 membered heteroaryl, each of which is optionally and independently substituted with C₁₋₆ alkyl, halo or OH.

24. (canceled)

25. (canceled)

26. The compound or salt thereof according to claim 1, wherein R² is a methyl.

27. (canceled)

28. The compound or salt thereof according to claim 23, wherein R² is a C₁₋₆ alkyl, substituted with —N(CH₃)₂.

29. The compound or salt thereof according to claim 23, wherein R² is a C₁₋₆ alkyl, substituted with morpholine or N-methylpiperizine.

30. (canceled)

31. The compound or salt thereof according to claim 1, wherein the compound is selected from:

| | |
|---|---|
| 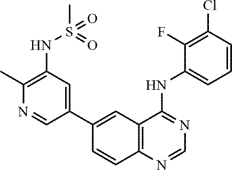 [SMILES: Cc1ncc(-c2ccc3ncnc(Nc4cccc(Cl)c4F)c3c2)cc1NS(C)(=O)=O] | 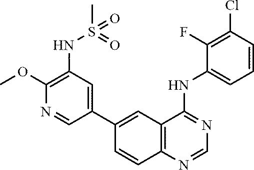 [SMILES: COc1ncc(-c2ccc3ncnc(Nc4cccc(Cl)c4F)c3c2)cc1NS(C)(=O)=O] |
| Compound 1 | Compound 2 |
| 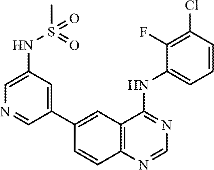 [SMILES: CS(=O)(=O)Nc1cncc(-c2ccc3ncnc(Nc4cccc(Cl)c4F)c3c2)c1] | 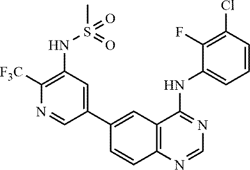 [SMILES: CS(=O)(=O)Nc1cc(-c2ccc3ncnc(Nc4cccc(Cl)c4F)c3c2)cnc1C(F)(F)F] |
| Compound 3 | Compound 4 |
| 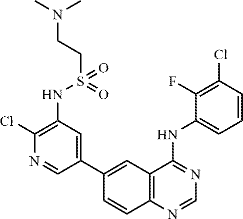 [SMILES: CN(C)CCS(=O)(=O)Nc1cc(-c2ccc3ncnc(Nc4cccc(Cl)c4F)c3c2)cnc1Cl] | 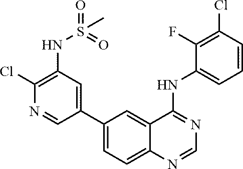 [SMILES: CS(=O)(=O)Nc1cc(-c2ccc3ncnc(Nc4cccc(Cl)c4F)c3c2)cnc1Cl] |
| Compound 5 | Compound 6 |
| 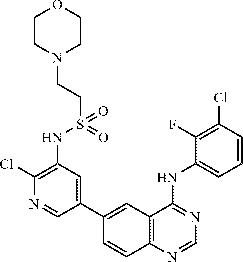 [SMILES: O=S(=O)(CCN1CCOCC1)Nc1cc(-c2ccc3ncnc(Nc4cccc(Cl)c4F)c3c2)cnc1Cl] | 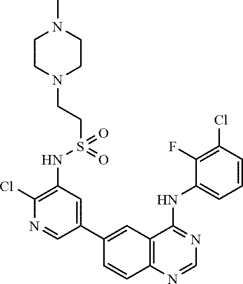 [SMILES: CN1CCN(CCS(=O)(=O)Nc2cc(-c3ccc4ncnc(Nc5cccc(Cl)c5F)c4c3)cnc2Cl)CC1] |
| Compound 7 | Compound 8 |
| 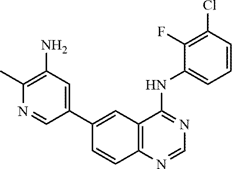 [SMILES: Cc1ncc(-c2ccc3ncnc(Nc4cccc(Cl)c4F)c3c2)cc1N] | 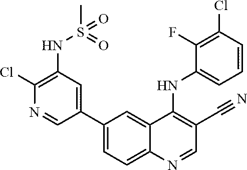 [SMILES: CS(=O)(=O)Nc1cc(-c2ccc3ncc(C#N)c(Nc4cccc(Cl)c4F)c3c2)cnc1Cl] |
| Compound 9 | Compound 10 |

.

32. A pharmaceutical composition comprising a compound, or salt thereof, according to claim 1, and a pharmaceutically acceptable excipient.

33. A method of treating, ameliorating, or preventing a EGFR and/or PI3K mediated disease or condition in a patient, comprising administering to said patient a therapeutically effective amount of a compound, or salt thereof, according to claim 1.

34. (canceled)

35. The method of claim 33, wherein said disease or condition is cancer.

36. The method of claim 35, wherein said cancer is selected from NSCLC, head & neck cancer, glioblastoma multiform, colorectal cancer, or a combination thereof.

37. The method of claim 33, wherein said patient is a human patient.

38. (canceled)

39. (canceled)

40. (canceled)

41. A kit comprising a compound, or salt thereof, according to claim 1, and instructions for administering said compound to a patient having a EGFR and/or PI3K mediated disease or condition.

42. (canceled)

43. (canceled)

44. (canceled)

45. (canceled)
